(19) United States
(12) Patent Application Publication
(10) Pub. No.: US 2025/0134578 A1
(43) Pub. Date: May 1, 2025

(54) ELECTROSURGICAL INSTRUMENT

(71) Applicant: Creo Medical Limited, Chepstow, Gwent (GB)

(72) Inventors: George Christian ULLRICH, Anglesey (GB); David Edward WEBB, Bangnor (GB); Christopher Paul HANCOCK, Chepstow (GB); Aeron JONES, Chepstow, Gwent (GB)

(21) Appl. No.: 18/722,504

(22) PCT Filed: Oct. 13, 2022
(86) PCT No.: PCT/EP2022/078548

§ 371 Date: Jun. 20, 2024

(30) Foreign Application Priority Data
Dec. 24, 2021 (GB) 2119000.4

(51) Int. Cl. A61B 18/14; A61B 18/00

(52) U.S. Cl. CPC A61B 18/14; A61B 2018/00077; A61B 2018/1435

(57) ABSTRACT

An electrosurgical instrument for applying radiofrequency or microwave frequency energy to biological tissue. The instrument includes a tip with a planar body separating two conductive elements on opposite surfaces. It also includes a coaxial feed cable with an inner and outer conductor, separated by a dielectric material, for conveying a working signal. The inner conductor connects to the first conductive element, and the outer to the second, enabling the tip to receive the signal. The first conductive element has a pattern to set its electrical length greater than a reference instrument tip. The reference tip has a planar body of the same shape and dimensions, separating two conductive elements covering opposite surfaces.

### FIELD OF THE INVENTION

The present invention relates to electrosurgical instruments for delivering electromagnetic energy (e.g. radiofrequency and/or microwave energy) into biological tissue for cutting tissue and/or for haemostasis (i.e. promoting blood coagulation). For example, the invention may be applied to instruments sized to be suitable for insertion through the instrument channel of a standard surgical endoscope.

### BACKGROUND

Surgical resection is a means of removing sections of organs from within the human or animal body. Such organs may be highly vascular. When tissue is cut (divided or transected) small blood vessels called arterioles are damaged or ruptured. Initial bleeding is followed by a coagulation cascade where the blood is turned into a clot in an attempt to plug the bleeding point. During an operation, it is desirable for a patient to lose as little blood as possible, so various devices have been developed in an attempt to provide blood free cutting. For endoscopic procedures, bleeds are also undesirable, and need to be dealt with in an expedient manner, since the blood flow may obscure the operator's vision, which may prolong surgery and potentially lead to the procedure needing to be terminated and another method used instead, e.g. open surgery.

Electrosurgical generators are prevalent in hospital operating theatres, often for use in open and laparoscopic procedures, and increasingly for use with surgical scoping devices, e.g. an endoscope or the like. In endoscopic procedures the electrosurgical accessory is typically inserted through a lumen inside an endoscope. Considered against the equivalent access channel for laparoscopic surgery, such a lumen is comparatively narrow in bore and greater in length.

Instead of a sharp blade, it is known to use radiofrequency (RF) energy to cut biological tissue. The method of cutting using RF energy operates using the principle that as an electric current passes through a tissue matrix (aided by the ionic contents of the cells and the intercellular electrolytes), the impedance to the flow of electrons across the tissue generates heat. In practice, an instrument is arranged to apply an RF voltage across the tissue matrix that is sufficient to generate heat within the cells to vaporise the water content of the tissue. However, as a result of this increasing desiccation, particularly adjacent to the RF emitting region of the instrument (which has the highest current density of the current path through tissue), direct physical contact between the tissue and instrument can be lost. The applied voltage then manifests itself as a voltage drop across this small void, which causes ionisation in the void that leads to a plasma. Plasma has a very high volume resistivity compared with tissue. The energy supplied to the instrument maintains the plasma, i.e. completes the electrical circuit between the instrument and the tissue. Volatile material entering the plasma can be vaporised and the perception is therefore of a tissue dissecting plasma.

GB 2 523 246 describes an electrosurgical instrument for applying to biological tissue RF electromagnetic energy and/or microwave frequency EM energy. The instrument comprises a shaft insertable through an instrument channel of a surgical scoping device. At a distal end of the shaft there is an instrument tip comprising a planar transmission line formed from a sheet of a first dielectric material having first and second conductive layers on opposite surfaces thereof. The planar transmission line is connected to a coaxial cable conveyed by the shaft. The coaxial cable is arranged to deliver either microwave or RF energy to the planar transmission line. The coaxial cable comprises an inner conductor, an outer conductor coaxial with the inner conductor, and a second dielectric material separating the outer and inner conductors, the inner and outer conductors extending beyond the second dielectric at a connection interface to overlap opposite surfaces of the transmission line and electrically contact the first conductive layer and second conductive layer respectively. The instrument further comprises a protective hull with a smoothly contoured convex undersurface facing away from the planar transmission line. The undersurface comprises a longitudinally extending recessed channel formed therein. A retractable needle is mounted within the instrument, and operable to extend through the recessed channel to protrude from a distal end of the instrument. The needle can be used to inject fluid into a treatment zone before the RF or microwave energy is applied.

The present invention has been devised in light of the above considerations.

### SUMMARY OF THE INVENTION

The present invention provides a development to the concept discussed in GB 2 523 246.

It would be desirable to reduce the size of the instrument, e.g. by making it thinner and/or shorter. A compact arrangement may provide several advantages. For example, a compact arrangement may allow the instrument to be used within narrower scoping devices and/or in smaller biological structures, may enable the instrument to be more easily manoeuvred, and/or may help to improve the control and precision at the instrument tip.

However, it is difficult to reduce the size of the instrument whilst retaining its functionality.

For example, the ability to reduce the instrument size is limited by the size of the planar transmission line at the instrument tip. As discussed in GB 2 523 246, the overall length of the planar transmission line arrangement is important in terms of matching the impedance (or the energy delivery) of (or from) the coaxial transmission line with (or into) the biological tissue, i.e. the structure may form a quarter wave impedance transformer or a half wavelength resonator.

In order to efficiently transfer energy into tissue, the instrument tip may be configured with a physical length that corresponds to a particular electrical length (i.e. number of wavelengths) at a desired frequency of energy. For example, in order to provide efficient energy transfer at 5.8 GHz microwave energy, the physical length of the instrument tip may be selected to correspond to a half wavelength at that frequency (taking into account the dielectric constant of the material), for the instrument tip to act as a half-wavelength resonator. However, since the physical length of the instrument tip is selected to provide a particular electrical length (i.e. to correspond to a certain number of wavelengths at the desired frequency), the physical length of the instrument tip cannot be reduced whilst retaining the desired electrical properties. Instead, as the physical length is reduced, the electrical length will also be reduced, resulting in destructive reflections at the interface with biological tissue at the desired frequency (e.g. 5.8 GHz), and causing reduced efficiency of energy delivery through the instrument tip. The ability to reduce the size of the instrument tip has therefore previously been limited by the need for efficient energy transfer.

The inventors have developed a modified instrument which can effectively decouple the instrument tip's physical length from its electrical length. The instrument may therefore provide efficient energy transfer into tissue at a predetermined treatment frequency (e.g. microwave frequency) which may be the same frequency as used in the prior art, whilst having different (e.g. smaller) dimensions than the prior art.

A first aspect of the invention provides an electrosurgical instrument for applying radiofrequency (RF) electromagnetic (EM) energy and/or microwave frequency EM energy to biological tissue, the instrument comprising: an instrument tip comprising a planar body separating a first conductive element on a first surface thereof from a second conductive element on a second surface thereof, the second surface facing in the opposite direction to the first surface; a coaxial feed cable comprising an inner conductor, an outer conductor coaxial with the inner conductor and a dielectric material separating the inner and outer conductors, the coaxial feed cable being for conveying a working signal comprising an RF signal and/or microwave signal; wherein the inner conductor is electrically connected to the first conductive element and the outer conductor is electrically connected to the second conductive element to enable the instrument tip to receive the working signal, wherein the first conductive element comprises a pattern configured to set an electrical length of the instrument tip to be greater than an electrical length of a reference instrument tip, wherein the reference instrument tip comprises a reference planar body separating a reference first conductive element entirely covering a reference first surface thereof from a reference second conductive element entirely covering a reference second surface thereof, the reference second surface facing in the opposite direction to the reference first surface, wherein the reference planar body has the same shape and dimensions as the planar body.

By patternising the first conductive element in this manner, the instrument tip may be made smaller than those of the prior art whilst maintaining the ability to efficiently transfer the working signal into tissue.

As used herein, the phrase “working signal” may refer to a signal for treating and/or diagnosing biological tissue. For example, the working signal may be a treatment signal for coagulating and/or ablating tissue. Additionally or alternatively, the working signal may be a diagnostic signal for diagnosing tissue, for example, detecting a tissue type based on how it reflects energy. In an embodiment, the working signal is an RF EM signal and/or a microwave frequency EM signal.

As used herein, the phrase “electrical length” may refer to a length of the instrument tip as calculated using the working signal's wavelength 1, i.e. it may refer to the length of the instrument tip as “seen” by the working signal. The electrical length may be calculated as a fraction or multiple of wavelengths. For example, in order to act as a half wave resonator, the instrument tip may have an electrical length of approximately ½λ. This can be calculated as

$$\frac{1}{2} λ = \frac{1}{2} \frac{c}{f \sqrt{{ε}_{eff}}} ,$$

where c is the speed of light and ε_{eff} is the effective dielectric constant of the planar body. The effective dielectric constant may depend on the thickness of the conductive element, how close it is to an edge of the planar body, and the material surrounding the conductive element. The effective dielectric constant may vary along the length of the conductive element, and therefore using one value for ε_{eff} in this formula may be an approximation. For example, to resonate at a half wavelength at a frequency of 5.8 GHz and using a planar body comprising a ceramic dielectric material such as alumina having a dielectric constant of approximately 9.8, the instrument tip may have an electrical length close to

$$\frac{1}{2} λ = \frac{1}{2} \frac{c}{f \sqrt{ε}} = 8.3 mm .$$

As used herein, the “reference instrument tip” is not part of the electrosurgical instrument. Rather, the reference instrument tip is used for reference to describe the advantages of the invention compared to an instrument which is otherwise equivalent to the claimed electrosurgical instrument, but which has a non-equivalent (reference) first conductive element that entirely covers an equivalent (reference) first surface of an equivalent (reference) planar body.

As used herein, the phrase “pattern” may refer to a shape and/or size of the first conductive element. The pattern may be considered relative to the planar body, e.g. so that the first conductive element has a shape and size that does not cover the entirety of a first surface of the planar body. The pattern provides the instrument tip with an electrical length that is greater than a reference electrical length which would otherwise be provided by the reference first conductive element covering the entirety of the (equivalent) reference first surface of the (equivalent) reference planar body. The pattern may therefore be considered to include an electrically lengthening section that increases the electrical length of the instrument tip relative to the electrical length of the reference instrument tip. The pattern may include one or more voids (i.e. the absence of a conductive material) to increase the impedance of the conductive element and thereby increase its electrical length.

Embodiments may therefore allow the physical size (e.g. length) of the instrument to be made smaller than prior art arrangements, whilst maintaining the ability to efficiently deliver the same working signal into tissue (e.g. a microwave frequency signal), since the pattern can increase the electrical length of the instrument and thereby offset any decreases in electrical length which would otherwise occur when decreasing the physical size the instrument.

As used herein, the “first surface” may refer to an upper surface of the instrument tip, and the “second surface” may refer to a lower surface of the instrument tip. The first and second conductive elements may each comprise a layer of metallisation formed on opposite surfaces of the planar body. The planar body, first conductive element, and second conductive element may together be considered to form a planar transmission line. Other features of the electrosurgical instrument may be understood in view of GB 2 523 246 and GB 2 503 673, which are incorporated herein by reference.

Preferably, the instrument tip is configured as a resonator having an electrical length corresponding to a fraction or multiple of wavelengths (e.g. half wavelength) at a desired frequency (e.g. at the frequency of input microwave and/or RF energy). By providing an instrument tip which acts as a resonator at the desired frequency, unwanted reflections can be reduced at the interfaces between the instrument tip and coaxial cable and/or tissue, thereby helping to efficiently deliver energy from the coaxial cable into the tissue. Additionally, by providing an instrument tip which acts as a resonator, it can be ensured that the instrument tip transmits the working signal as a standing wave having a maximum at a distal end of the instrument tip, to deliver the maximum available amount of energy into the tissue.

The invention may be further understood in view of the following theory.

In general, the resonant frequency f of a resonant circuit is related to its inductance L and capacitance C as follows:

$$f = \frac{1}{2 π \sqrt{L C}} \qquad ( equation 1 )$$

In order to efficiently transfer the working signal into tissue, it is preferable for the instrument tip to have a resonant frequency which is close or equal to the frequency of the working signal.

The resonant frequency of a circuit is also related to the wavelength 2 of the signal, the speed of light c, and the dielectric constant ε as follows:

$$λ = \frac{c}{f \sqrt{ε}} \qquad ( equation 2 )$$

By patterning the first conductive element, its inductance L and/or capacitance C may be varied (increased) to provide a desired electrical length, rather than requiring the instrument tip as a whole to be physically increased in size (e.g. lengthened and/or widened) in order to increase the electrical length. The pattern may therefore include one or more inductive elements and/or capacitive elements configured to electrically lengthen the instrument tip relative to the reference instrument tip, to provide a desired electrical length even at relatively small sizes.

As used herein, an “inductive element” may refer to any patterned conductive portion which has predominantly inductive (rather than capacitive) properties, e.g. a predominantly inductive contribution to impedance. An inductive element may also be referred to as an “inductive structure”, “inductive track” or “inductor”.

As used herein, a “capacitive element” may refer to any conductive portion which has predominantly capacitive (rather than inductive) properties, e.g. a predominantly capacitive contribution to impedance. A capacitive element may refer to only a part of a capacitor (e.g. only the portion on the first conductive element) rather than an entire capacitor (which would further include the planar body and second conductive element). A capacitive element may also be referred to as a “capacitive structure” or “capacitive plate”.

Optionally, inductive elements and capacitive elements may be visually distinct from each other. For example, inductive elements may be relatively thinner than capacitive elements to provide a relatively higher inductance and lower capacitance. For example, inductive elements may present as relatively thin conductive tracks, whereas capacitive elements may present as relatively larger conductive areas.

As will be further explained with reference to FIG. 2, when the instrument tip is configured as a resonator, particular regions of the instrument tip may be considered to act as inductors (where current is high and electric field is low) or as capacitors (where current is low and electric field is high). For example, in the case of a half wave resonator, the proximal and distal ends of the instrument tip may act as capacitors, and the centre may act as an inductor.

Some structures may be considered to provide both a capacitive and inductive effect, but may be characterised as being “predominantly” capacitive or inductive. For example, a thin track in the centre of a half wave resonator may be a type of inductive element, since it may contribute primarily to inductance rather than capacitance. However, the thin track may also contribute a (smaller) amount to capacitance, due to the underlying second conductive element which is spaced from the first conductive element by the planar body. Conversely, a conductive plate in a proximal or distal region of a half wave resonator may be a type of capacitive element, but may be modified in some embodiments to include a cut-out (void), so as to form a loop (thinned track) which may also be considered to contribute to inductance. The capacitive contribution of the loop may be greater at the distal and/or proximal regions of the instrument tip than nearer the centre.

Optionally, the pattern may be configured to set an impedance value of the instrument tip to match an impedance value of the coaxial cable. As used herein, an impedance “match” may allow a variation between ⅓ (33%) and 3 times (300%) the desired value, or optionally between 70% and 140% of the desired value. The instrument may therefore be very tolerant of varying loads. An impedance match can help to improve the efficiency of energy transfer into tissue. The pattern may include inductive and/or capacitive elements configured to provide the impedance match.

This may be further understood in view of the fact that the inductance L and capacitance C of a transmission line are related to its impedance Z as follows:

$$Z = \sqrt{\frac{L}{C}} \qquad ( equation 3 )$$

The pattern of the first conductive element may be therefore configured (by varying its inductance L and/or capacitance C) to provide a desired impedance Z.

In use, the efficiency of energy transfer may be affected by the impedance match at the coaxial cable and the impedance match at the tissue, since there will be reflections at both interfaces. However, the tissue's impedance can vary based on the type of tissue being contacted, or the amount (e.g. width) of tissue being contacted. For example, the impedance of tissue could vary between approximately 20 to 300 ohms. Conversely, the impedance of the coaxial cable could be constant e.g. 50 ohms. It may therefore be particularly useful to provide the instrument tip with an impedance which is matched to that of the coaxial cable. In this way, signal reflections cancel out. For example, reflections from a junction between the tissue and instrument tip cancel reflections from a junction between the instrument tip and tissue.

Advantageously, the instrument tip may also be configured as a resonator to improve energy transfer, as discussed above. By configuring the instrument tip as a resonator, the voltages at the distal end of the instrument tip increase, and the currents decrease, so that power passes readily from the coaxial line through the instrument tip into the tissue. For this reason, the resonant effect of the instrument tip can play a significant role in the efficiency of energy transfer. Optionally, configuring the pattern to provide (or improve) an impedance match to the coaxial feed cable help to further improve the efficiency of this energy transfer, by effectively increasing the amplitude of the working signal within the instrument tip.

In general, adjusting the pattern to provide an impedance match may be particularly useful when trying to make the instrument tip narrower (since narrowing the blade will not affect resonant length but will affect impedance), and configuring the pattern to provide a desired electrical length may be particularly useful when trying to make the active tip shorter (since this will affect resonant length as well as impedance matching, and resonance may have a more significant effect on energy delivery than impedance mismatch).

Optionally, the instrument tip is configured as a half wave resonator. The first conductive element may therefore be patterned to provide an electrical length corresponding to a half wavelength of the working signal. An instrument tip configured as a half wave resonator may be approximated as having three regions: a proximal capacitive region, a central inductive region, and a distal capacitive region. The first conductive element may therefore be selectively patterned in one or more of these three regions to increase its impedance, by taking advantage of the capacitive or inductive properties in the different regions of the instrument tip to increase its electrical length.

In variant embodiments, the instrument tip could be configured differently. For example, the instrument tip could be configured as a quarter wave impedance transformer. Alternatively, the instrument tip could be configured to resonate at another fraction or multiple of wavelengths, e.g. based on the desired frequency and size of the instrument.

Optionally, the instrument tip is configured to resonate at one of the following predetermined frequencies: 915 MHz, 2.45 GHz, 5.8 GHz, 14.5 GHz, 24 GHz. These frequencies may be particularly useful for treating biological tissue. For example, the instrument tip may be configured as a half wave resonator at 5.8 GHz. For resonating at lower frequencies (e.g. 915 MHz), the instrument tip may include actual capacitors or inductors (referred to as “lumped components”), which have the potential to significantly shorten devices which may otherwise be too long at these frequencies.

Optionally, the pattern forms at least one inductive element located at a central zone of the instrument tip. The phrase “central zone” may refer to a zone between a proximal zone of the conductive element (near the coaxial feed cable) and a distal zone of the conductive element (further away from the coaxial feed cable). For example, optionally, the instrument tip comprises (e.g. consists of, is divided into) four quarters along its length, the middle two quarters being combined together to form a central zone, and wherein the pattern forms at least one inductive element in the central zone. In an embodiment, each quarter has substantially the same length (e.g. the quarters are equal quarters). The distal quarter may be referred to as the distal zone and/or the proximal quarter may be referred to as the proximal zone. The zones of the instrument tip may be formed along a length of the planar body, optionally further including a spacer element (discussed further below) if present.

The inductive element may be shaped to increase the impedance in the central zone, and thereby cause the instrument tip to appear electrically lengthened. Providing an inductive element in the central zone may be especially advantageous since the central zone may then contribute primarily to the inductance (rather than capacitance) of the instrument tip when the instrument tip is configured as a half wave resonator.

In a resonator, the current may be highest in the central zone of the conductive element, and may be lowest at the edges. Accordingly, since inductance is related to current, by providing the inductive element in the centre, it may have the largest effect on improving the efficiency of energy delivery.

In variant embodiments, the central zone of the first conductive element may include a capacitive element, and the inductive element may be provided in a different zone of the instrument tip (e.g. at the distal or proximal zone).

The inductive element may comprise a thin track. As used herein, the phrase “thin track” may refer to a conductive section (region) that is relatively thinner than one or more other sections (regions) of the first conductive element (e.g. relatively thinner than a proximal and/or distal section, e.g. relatively thinner than the one or more capacitive elements). Optionally, the inductive element may comprise a first conductive region having a width being less than or equal to 90% of a width (e.g. maximum width) of a second conductive region of the first conductive element, the second conductive region being located in the distal or proximal zone. Similarly, the width of the first conductive region may also be less than or equal to 90% of a width (e.g. maximum width) of a third conductive region of the first conductive element, the third conductive region being located in the other of the proximal or distal zone.

Optionally, the width of the first conductive region may be less than or equal to 80% of the width of the second and/or third conductive region, optionally less than or equal to 70%, optionally less than or equal to 60%, optionally less than or equal to 50%, optionally less than or equal to 40%, optionally less than or equal to 30%, optionally less than or equal to 20%, optionally less than or equal to 10%. By increasing the difference in relative widths between the conductive regions, their relative inductive and capacitive effects can be increased.

Optionally, the inductive element may comprise a conductive region having a width being less than or equal to 90% of a maximum width of the planar body in the central zone. Optionally, the width may be less than or equal to 80% of the maximum width of the planar body in the central zone, optionally less than or equal to 70%, optionally less than or equal to 60%, optionally less than or equal to 50%, optionally less than or equal to 40%, optionally less than or equal to 30%, optionally less than or equal to 20%, optionally less than or equal to 10%. The absolute width of the inductive element may be selected to provide a desired impedance.

Thinning the conductive track is a particularly convenient method of increasing its inductance (and thus altering its electrical length and/or impedance match) without requiring the physical size of the instrument to be increased.

In some embodiments, the pattern may include a single inductive element in the central zone. For example, the pattern may (only) include a single inductive element that extends along a longitudinal (e.g. central) axis of the instrument tip. The inductive element may connect a proximal conductive region (e.g. capacitive plate) in a proximal zone of the active tip with a distal conductive region (e.g. capacitive plate or conductive loop) in a distal zone of the active tip. Preferably, the inductive element has a length configured to prevent the working signal from coupling across a gap adjacent the inductive element and between the proximal and distal conductive regions. For example, the gap may have a length (as measured between different conductive regions, e.g. the proximal and distal conductive regions) that is greater than or equal to twice the thickness of the planar body. For example, the planar body may have a thickness of 0.5 mm, and the gap(s) between conductive regions may have a length of greater than or equal to 1 mm. The reasoning for this is that the EM fields may spread out from beneath the conductive regions for a distance determined by the thickness of the planar body (e.g. first dielectric material). When the fields of two conductive regions overlap substantially, there may be coupling between the conductive regions and across the length of the gap. By providing the pattern with one or more gaps between inductive and/or conductive regions that are greater than or equal to twice the thickness of the planar body, it can be ensured that the working signal is limited to following the path provided by the conductive element.

In variant embodiments, the gap may be configured to have a different length (e.g. smaller than twice the thickness of the planar body), depending on the surrounding pattern.

As used herein, “longitudinal” may refer to a direction extending along the planar body and between the proximal and distal ends of the instrument tip, and “lateral” may refer to a direction extending across the planar body and transversely to the longitudinal direction. A “longitudinal” extension may include a curve or taper, e.g. if the planar body is curved or tapered, but may be considered to be ‘predominantly’ extending in a proximal/distal direction rather than a lateral direction.

In some embodiments, the pattern may include the longitudinal inductive element in addition to one or more lateral inductive elements, e.g. a pair of inductive elements which branch (fork) from the longitudinal inductive element and extend laterally (e.g. transversely, perpendicularly) from it toward an edge of the planar body. The lateral inductive elements may also be referred to herein as “laterally extending arms”. At or near the edge of the planar body, the pattern may divert again towards a longitudinal direction, to provide a further pair of longitudinal inductive elements, each of which extends from a respective lateral inductive element towards a distal end of the instrument tip. The pairs of lateral and longitudinal inductive elements may form part of a conductive loop (e.g. a D-shaped loop) at a proximal end of the instrument tip, with the inductive elements forming the parts of the loop that lie in the central zone of the instrument tip.

In some embodiments, the pattern may omit the first (more proximal) longitudinal inductive element discussed above. Accordingly, the one or more lateral inductive elements may extend directly from a capacitive plate in a proximal or (in part) central zone of the instrument tip. The pattern may therefore comprise a conductive loop at a distal portion of the instrument tip, the conductive loop being connected (e.g. directly connected) to a capacitive element at a proximal portion of the instrument tip. By omitting the central longitudinal track, similar electrical lengths may be achieved as discussed above, whilst also allowing an even smaller physical size to be obtained.

The portion of the conductive loop that lies in the central zone may effectively provide a pair (e.g. an opposing pair) of longitudinal (and optionally lateral) inductive elements. The conductive loop may therefore contribute significantly to the inductance of the instrument tip, e.g. since it may include a plurality of inductive elements along opposite sides of the instrument tip, rather than only a single inductive element in a centre of the instrument tip.

Optionally, the pattern includes a lengthening section configured to increase the physical length of the first conductive element relative to a physical length of the planar body. By increasing the physical length of the conductive element, the lengthening section may effectively also increase the length of the first conductive element as seen by the working signal, thereby also increasing the electrical length without requiring the physical length of the instrument tip (e.g. planar body) to also be increased.

The lengthening section may include one or more conductive sections that are misaligned with a length direction (longitudinal direction) of the planar body. Optionally, the lengthening section may include a non-linear track (e.g. thin track) for conveying the working signal in a direction transverse to or away from a distal end of the instrument tip. The non-linear track may effectively provide a non-linear (indirect) pathway for the working signal (compared to the reference conductive element which may be considered to provide a ‘linear’ or ‘direct’ track to the distal end). For example, the lengthening section may include one or more deflections, i.e. one or more points where the conductive element (e.g. thin track) changes direction (so as not to form a single straight line). For example, the lengthening section may include one or more deflections in the form of one or more bends, corners, and/or curves. The lengthening section may include one or more straight portions separated from another section by a deflection.

Optionally, the lengthening section may have an undulating shape (e.g. a serpentine, sinusoidal, wavy, zig-zag, or square wave shape). Preferably, adjacent portions of the lengthening section (e.g. adjacent undulations) are spaced by gaps sized based on the wavelength of the working signal to be large enough to prevent (or reduce) the signal coupling across the gaps, to thereby ensure that the input energy travels through the full length of the lengthening section.

Alternatively or in combination, the lengthening section may include one or more branches extending along a respective one or more peripheral edges of the planar body. Since the branches are located along peripheral edges of the distal zone, they may be useful for applying energy into the tissue (e.g. to perform cutting and/or coagulation). The one or more branches may extend from a distal portion of the conductive element proximally towards (or into/through) the central zone, to add extra physical length to the track formed by the first conductive element (and thereby also increase its electrical length). The one or more branches may each terminate in a free end (in or near the central zone), e.g. they may be unconnected at their terminal ends from any other conductive structure. This arrangement may be considered to include both a capacitive element (e.g. in the distal zone) and an inductive element (e.g. in the central zone).

Optionally, the pattern forms at least one capacitive element in a distal quarter of the instrument tip. In this context, the at least one capacitive element may be referred to as a “distal capacitive element”.

Optionally, the pattern forms at least one capacitive element in a proximal quarter of the instrument tip. In this context, the at least one capacitive element may be referred to as a “proximal capacitive element.”

In a resonator (e.g. a half wave resonator), the proximal and distal regions are where the electric field is highest and thus have the largest effect on capacitance. Capacitive elements in these regions may therefore be particularly advantageous in these regions. Optionally, the pattern may include a distal capacitive element and a proximal capacitive element which are connected by a (central) inductive element.

In variant embodiments, the first conductive element may include a capacitive element in another region (e.g. the central region).

Optionally, the or each capacitive element comprises a conductive region having an area which fills a majority or an entirety of the respective quarter in which the capacitive element is formed. This may help to provide the maximum capacitance in these regions, to provide the desired electrical length and/or impedance match with the coaxial cable.

Optionally, the distal capacitive element extends along one or more peripheral edges in the distal quarter of the instrument tip (e.g. along a curved edge of the instrument tip). The distal capacitive element may therefore be in close contact with tissue to treat the tissue.

Optionally, a proximal section of the first conductive element (e.g. the proximal capacitive element) may be spaced from one or more lateral edges of the planar body in the proximal quarter of the instrument tip. The proximal section of the first conductive element may therefore avoid interaction with tissue, so that the energy can be primarily transmitted into tissue at the distal end of the instrument tip.

Optionally, the first and/or second conductive elements may be set back from the lateral edges of the planar body, in a proximal region of the instrument tip (e.g. proximal quarter of the instrument tip), e.g. by a distance of at least 0.1 mm, optionally at least 0.15 mm, optionally at least 0.2 mm along the proximal 3.5 mm of the tip. This may help to ensure that RF energy does not cut tissue in the proximal region of the instrument tip, and that the RF energy is instead distributed from the distal tip of the planar body. This may also provide a similar effect for microwave energy, although to a lesser extent.

Optionally, the pattern comprises a conductive loop encircling a non-conductive region, the conductive loop being located in a distal two quarters of the instrument tip. The non-conductive region may also be referred to as a “cut-out”, a “non-capacitive region”, a “void”, or a “hollow” area of a capacitive plate. By configuring the pattern in the distal two quarters as a conductive loop (rather than e.g. a conductive plate covering the entirety of the distal two quarters), the inductance and capacitive properties can be further tuned to provide a desired electrical length and/or impedance match. In particular, since the cut-out reduces the area of the conductive element, it can be used to reduce the capacitance. Additionally, since the loop may be considered to be a thin track (part of which may be located in the central region, i.e. in the second-distalmost), it may increase an inductance of the instrument tip. The loop may therefore be considered to include both a capacitive and inductive element.

Optionally, the conductive loop is configured to extend along one or more edges of the planar body at the distal zone of the instrument tip. The loop may therefore be used to impact and treat or diagnose tissue, in a similar manner as the distal capacitive element discussed above. As a further advantage, the loop may also provide a clearer and more easily visible working surface for clinicians, compared to e.g. a solid capacitive plate.

Optionally, the pattern is symmetrical about a longitudinal axis of the instrument tip. The “longitudinal axis” refers to an axis extending from the proximal zone to the distal zone of the instrument tip. By providing a first conductive element which is symmetrical across this axis, the instrument tip may provide a relatively symmetrical energy distribution across both sides of the instrument. Accordingly, the instrument may be more convenient to use, since the clinician need not worry about how to orientate the device in order to best convey the energy into tissue.

The instrument tip's second conductive element may be configured similarly to the first conductive element, e.g. having the same pattern as the first conductive element, e.g. so that conductive portions of the first and second conductive elements overlay each other. Alternatively, the second conductive element may be configured differently from the first conductive element. For example, the second conductive element may comprise a substantially uniform metallisation across the second surface of the planar body. The uniform metallisation may cover substantially all of the planar body (e.g. an underside thereof). Accordingly, the second conductive element may cover a majority or an entirety of the second surface of the planar body. This may provide a relatively convenient structure for the second surface of the planar body.

Optionally, the instrument tip may include a spacer element proximal to the planar body. The inner conductor may be electrically connected to the first conductive element by a proximal transmission line which overlies the spacer element. The first conductive element and/or second conductive element may be set-back from a proximal edge of the spacer element, optionally by a distance of at least 0.2 mm, optionally at least 0.3 mm, optionally at least 0.4 mm, optionally at least 0.5 mm, optionally at least 0.6 mm. Optionally, the spacer element may be devoid of the first conductive element. By setting back the first and/or second conductive elements from the proximal edge of the spacer element, this may help ensure that the first (upper/top) conductive layer is isolated from the outer conductor of the coaxial cable.

Optionally, the spacer element may be chamfered (as also discussed with respect to the third aspect, below). The chamfer may help to reduce the extent by which the proximal transmission line protrudes above the spacer element, thereby allowing the size of the instrument tip to be reduced. The instrument tip (e.g. the pattern of the first conductive element) may be configured to account for possible effects of this chamfer on the impedance and/or electrical length. For example, the shape and length of a junction between the proximal transmission line and the first conductive element may be configured (patterned) to improve microwave performance, since it is the reflection from this point which ideally cancels out with a reflection from the distal tip of the instrument tip to provide improved efficiency. For example, the size and shape of a proximal portion of the first conductive element may be patterned to provide an improved electrical length and/or impedance match.

The spacer element may be integrally formed with the planar body, or otherwise attached to a proximal edge of the planar body. The spacer element may comprise the same material as the planar body.

Optionally, the instrument tip may have a rectangular shape. Optionally, the instrument tip may have a non-rectangular shape. For example, a distal end of the planar body may be tapered (e.g. distally tapered or curved). For example, a width of the instrument tip in a plane of the planar body may progressively shorten when moving in a distal direction. This may be useful to assist in a cutting function of the instrument tip. However, a curved distal end may effectively decrease the electrical length of the instrument tip, e.g. relative to a rectangular planar body having the same length and width, since it effectively removes capacitance from the distal end of the instrument. This is further described in GB 2 503 673. In order to compensate for this decrease in electrical length, prior art arrangements increased the physical size of the instrument. Advantageously, the instrument tips of the present invention may be smaller than those arrangements, since the pattern of the first conductive element can help to offset the electrically shortening effect of the curved distal end. The planar body may therefore advantageously be made shorter than that of the prior art, whilst retaining its other functionalities.

In some embodiments, the pattern may (only) partially offset the electrically shortening effect of the curved distal end. The physical length of the instrument tip may therefore be less than those of the prior art, but the physical length may still be greater than the electrical length.

In some embodiments, the pattern may (fully) offset the effect of the curved distal end (or any other electrically shortening pattern), so that the physical length of the instrument may be made equal to the electrical length.

In some embodiments, the pattern may offset the effect of the curved distal end (or any other electrically shortening pattern) to such an extent that the physical length of the instrument tip may be less than its electrical length.

For example, in an arrangement having one or more electrically lengthening sections and one or more electrically shortening sections, the one or more electrically lengthening sections may be configured to cancel out and/or overcome the effect of the electrically shortening sections on the electrical length.

Accordingly, the pattern may set an electrical length of the instrument tip to be greater than or equal to a physical length of the instrument tip. Optionally, two or more quarters of the electrosurgical instrument may combine to provide an electrical length which is greater than their physical length.

Optionally, the instrument tip may have maximum a length of 10.0 mm or less. For example, the instrument tip may have a maximum length of less than 9 mm. For example, the instrument tip may have a maximum length of 8 mm. In this context, the length of the instrument tip may refer to a length of the planar body, optionally further including a spacer element if present. Therefore, the planar body may be made relatively shorter than previous arrangements, and may even be made shorter than its electrical length.

Optionally, the planar body may have a maximum width of 1.9 mm or less. For example, the planar body may have a maximum width of 1.8 mm. The planar body may therefore be relatively narrower than previous arrangements. The narrowing of the device may cause variations in impedance matching, e.g. by reducing the capacitance of the conductive element. However, the pattern may be configured to account for these variations, as discussed above, e.g. to provide an impedance match with the coaxial cable (e.g. 50 ohm impedance). The planar body may include deviations in width along its length (e.g. having a tapering distal region).

Preferably, the planar body may have a length that is greater than the maximum width.

Preferably, the planar body may have a maximum thickness of 0.5 mm or less.

The first conductive element may have a height that is more than several skin depths. The height required is proportional to the inverse square root of frequency. For example, for a conductive element formed of copper, the first conductive element may have a height of at least 5 microns (0.005 mm) for a working signal at 5.8 GHz, and at least 0.013 mm for a working signal at 915 MHz. Optionally, the first conductive layer may have a height of 0.05 mm or less, optionally 0.03 mm or less.

Preferably, the coaxial cable may have an outer diameter of 2 mm or less, more preferably 1.8 mm or less, more preferably 1.6 mm or less.

Optionally, the instrument tip may be configured for delivering fluid into biological tissue (e.g. using a needle or otherwise). Alternatively, optionally, the instrument tip may not be configured for delivering fluid into biological tissue, e.g. the instrument tip may be configured for (only) delivering energy into tissue.

Optionally, the instrument tip may include a nozzle at a distal end thereof for delivering pressurised fluid directly into biological tissue. The fluid may be injected into the biological tissue to raise the tissue into a bulge before treatment with a working signal (e.g. for cutting the tissue). By including a nozzle to deliver pressurised fluid, the surgical instrument may not require a needle to pierce the tissue. For example, the fluid pressure may be sufficiently high to pierce or penetrate through biological tissue (e.g. mucosa tissue and/or sub-mucosa tissue). Alternatively, the pressurised fluid may be used to perfuse or lift tissue in an area which has already been pierced (e.g. by another instrument or another needle on the surgical instrument which is not connected to the second fluid channel).

The nozzle may be positioned and shaped to avoid inadvertently piercing or otherwise damaging tissue. For example, the nozzle may have a relatively blunt or dull (non-sharp) fluid outlet for injecting the pressurised fluid into the tissue. For example, the nozzle may be cylindrical and/or may have a round (e.g. circular) outlet. The nozzle may be fixed (e.g. non-retractable) with respect to other elements of the instrument tip (e.g. with respect to the planar body). The nozzle may have an aperture that is flush with a surface of the instrument tip (e.g. a distal surface of the instrument tip) or is located proximal to a distal surface of the instrument tip, so as not to protrude from the instrument.

By providing a nozzle at a distal end of the instrument tip for delivering pressurised fluid directly into the biological tissue, the instrument may not require a retractable needle for piercing the biological tissue and conveying the fluid into the biological tissue. Further, the flexible shaft connected to the instrument tip may not require a push rod, control wire, or other means for controlling deployment of such a retractable needle at the instrument tip. The instrument may therefore have a relatively simpler configuration and smaller profile than prior art instruments requiring a retractable needle.

In some arrangements, in addition to (or instead of) the conductive pattern, the instrument tip may be modified in other manners to provide the appropriate electrical length and/or impedance match. For example, the capacitance could be modified by modifying the height of the planar body (thereby altering the distance between the first and second conductive elements). Alternatively or in combination, the inductance could be modified by or by changing the magnetic properties of the planar body. For example, in some embodiments, the planar body may comprise (e.g. consist of) a dielectric material (e.g. alumina). Additionally or alternatively, in some embodiments, the planar body may comprise a ferrite material (e.g. in at least one quarter of the planar body, e.g. in the central zone). This may provide for a change in inductance without requiring a thin conductive track.

Another aspect of the invention presents an electrosurgical instrument for applying radiofrequency (RF) electromagnetic (EM) energy and/or microwave frequency EM energy to biological tissue, the instrument comprising: an instrument tip comprising a planar body separating a first conductive element on a first surface thereof from a second conductive element on a second surface thereof, the second surface facing in the opposite direction to the first surface; a coaxial feed cable comprising an inner conductor, an outer conductor coaxial with the inner conductor and a dielectric material separating the inner and outer conductors, the coaxial feed cable being for conveying a working signal comprising an RF signal and/or microwave signal; wherein the inner conductor is electrically connected to the first conductive element and the outer conductor is electrically connected to the second conductive element to enable the instrument tip to receive the working signal, wherein the first conductive element comprises a pattern configured to set an electrical length of the instrument tip to be greater than or equal to a physical length of the instrument tip.

The further features and advantages presented in respect of the first aspect are equally applicable to and are restated in respect of this other aspect.

A second aspect of the invention presents an alternative solution for providing a shorter instrument tip. Similarly to the first aspect, the second aspect also provides a first conductive element that is patterned to mitigate destructive reflections at an end of the conductive element and thereby ensure efficient energy transfer into tissue. However, the second aspect achieves this in a different manner from the first aspect.

The second aspect of the invention provides an electrosurgical instrument for applying radiofrequency (RF) electromagnetic (EM) energy and/or microwave frequency EM energy to biological tissue, the instrument comprising: an instrument tip comprising a planar body separating a first conductive element on a first surface thereof from a second conductive element on a second surface thereof, the second surface facing in the opposite direction to the first surface; a coaxial feed cable comprising an inner conductor, an outer conductor coaxial with the inner conductor and a dielectric material separating the inner and outer conductors, the coaxial feed cable being for conveying a working signal comprising an RF signal and/or microwave signal; and wherein the inner conductor is electrically connected to the first conductive element and the outer conductor is electrically connected to the second conductive element to enable the instrument tip to receive the working signal, wherein the first conductive element comprises an elongate track having a peripheral section for conveying the working signal around a periphery of a distal region of the instrument tip (e.g. the planar body) for contacting tissue. In an embodiment, the peripheral section has a physical length that is greater than a physical length (e.g. in a direction aligned with a longitudinal axis of the instrument tip) of the distal region.

As used herein, the phrase “distal region” may refer to a distalmost section of the instrument tip (e.g. planar body), e.g. a distal 10% of the length of the instrument tip, optionally at least (or about) 20%, optionally at least (or about) 30%, optionally at least (or about) 40%, optionally at least (or about) 50%.

As used herein, the phrase “around a periphery” may refer to a path that is located on top of the periphery of the distal region and follows in a direction along (around) the periphery of the distal region. This may be distinguished from a track that entirely covers the first surface, and which would instead convey the working signal directly from a proximal end of the instrument tip a distal end of the instrument tip (e.g. in a direction aligned with a longitudinal axis of the instrument tip), without conveying the signal around the periphery.

Because the peripheral section conveys the working signal around the periphery, the peripheral section of the conductive element may have a physical length that is greater than a physical length of the distal region of the first surface of the planar body (e.g. in a direction aligned with a longitudinal axis of the instrument tip).

By providing an elongate track that extends (at least in part) around the periphery of a distal region of the planar body, the energy density of the track for contacting tissue may be increased (e.g. relative to a reference first conductive element that covers an entire first surface of an (equivalent) reference planar body). Additionally, the physical length of the track in contact with tissue may be increased relative to the reference first conductive element. The elongate track may therefore help to efficiently transfer energy into tissue, by effectively promoting energy transfer (i.e. losses) from the track into the surrounding tissue as the working signal travels along the peripheral section.

In contrast to prior art arrangements, the first conductive element of the second aspect may not configure the instrument tip as a resonator. Instead, the conductive element may configure the instrument tip as a lossy transmission line at the working signal. A “lossy transmission line” is a term of the art which may denote a transmission line which is configured to promote signal losses when in contact with a load (e.g. tissue). A lossy transmission line may be configured to lose energy at a fairly steady rate along its length when in contact with tissue, and not to be particularly frequency sensitive, whereas a resonator may be configured to lose energy to tissue at particular points along its length, and not at others, such as to lose energy to tissue at the distal end, but not halfway along the blade, and to have a resonant frequency at which it is designed to work best.

By patterning the first conductive element in this manner (e.g. the pattern being in the form of the elongate track), as the working signal travels along the peripheral section and contacts tissue, energy may be efficiently absorbed into the tissue, thereby decreasing the amplitude of the signal along the length of the peripheral section. The amplitude may therefore be reduced to such an extent as to mitigate or reduce any reflections (and thus destructive interference) at a terminal end of the conductive element. As a result, the pattern may allow the instrument tip to have a different (e.g. smaller) size than the prior art, whilst also avoiding the effects of destructive interference that would otherwise occur when reducing the size of the prior art resonant instrument tips.

The length and/or width of the first conductive element (e.g. the elongate track or the peripheral section) may be modified (patterned) to further tune these effects and provide desired signal loss properties along the length of the elongate track, e.g. by increasing its inductance and/or impedance. For example, the elongate track may have a length and width configured so that, by the time the working signal reaches a terminal end of the conductive element, its amplitude will be significantly reduced (e.g. to less than 10% of an input amplitude at an input (proximal) end of the first conductive element, optionally, less than 5%, optionally zero), to thereby significantly reduce or eliminate any reflections and resulting interference at the terminal end of the conductive element.

The elongate track (e.g. peripheral section) may therefore be relatively long and/or thin compared to a length and/or width of the planar body. Optionally, the elongate track (e.g. the peripheral section) may have a width of less than 30% of a maximum width of the planar body (e.g. the distal region), optionally less than 20%, optionally less than 10%.

A narrow width may be particularly advantageous in the peripheral section of the elongate track, since narrowing the track in the peripheral section may increase the power density across the track, thereby ensuring that more power can be closer to the tissue and therefore transferred from the instrument into the tissue. In other words, as the track is narrowed, the working signal's power is distributed over a narrower width of track. By providing a narrow peripheral section (which is at a distal periphery for contacting tissue), a higher amount of power can be positioned close to the edge of the instrument (rather than distributed over a wider track). Therefore, by providing a narrow peripheral section, a greater amount of power can be efficiently transferred into tissue, thereby further reducing the signal amplitude within the track and reducing the likelihood of any reflections at its terminal end. The track width may be configured so that power is transferred into tissue at an optimum rate, so that neither all of it is used up in the first few mm nor is there too much left to be reflected at the end.

Optionally, the elongate track may also include a narrow proximal section, e.g. having a width of less than 50% of a maximum width of the planar body, optionally less than 40%, optionally less than 30%, optionally less than 20%, optionally less than 10%.

The width of the elongate track (e.g. peripheral section) may be substantially uniform, but may vary along its length, e.g. to account for curves and corners along the elongate track. The peripheral section may therefore have slight deviations in width, e.g. varying within ±10%, optionally within ±5%.

For example, the peripheral section may extend around at least a distal 30% of the planar body, optionally at least a distal 40% of the planar body, optionally at least a distal 50% of the planar body. In other words, the distal region may cover a distal 30% of the length of the planar body, optionally a distal 40% of the length of the planar body, optionally a distal 50% of the length of the planar body, and the peripheral section may extend around the entirety of the distal region. By lengthening the elongate track to extend around a relatively large distal region, the amount of energy transfer into tissue can be further increased.

Optionally, a distal end of the planar body may be curved, and the peripheral section may extend around a majority or an entirety of the curved distal end. The curved distal end may provide a useful blade for treating tissue

The elongate track may include a proximal section for connecting the coaxial feed cable to the peripheral section. Optionally, the proximal section may be set back (spaced) from one or more lateral edges of the planar body in a proximal region (e.g. proximal quarter or proximal half of the instrument tip (e.g. planar body). Similarly to the first aspect, this may help to ensure that energy is primarily transferred into tissue from the distal end (rather than the proximal end) of the instrument tip.

The proximal section may include an inductive element (e.g. a narrow track) which may include any of the features discussed above in relation to the first aspect. The proximal section may have a capacitive element (e.g. an enlarged proximal end) for connecting to the coaxial feed cable. The capacitive element may include any of the features discussed above in relation to the first aspect. The inductive and/or capacitive element may be sized to improve an impedance match to a coaxial cable and/or tissue (e.g. relative to a reference conductive element covering the entirety or majority of the proximal region of the planar body).

Optionally, a majority or entirety of the first conductive element may be formed of the elongate track. The first conductive element may comprise one or more elements which are configured similarly to the inductive elements discussed in the first aspect.

Optionally, the elongate track may further include a lengthening section to further increase the physical length of the elongate track relative to a physical length of the planar body. The lengthening section may therefore help increase the length (and therefore losses) through the transmission line, without requiring extra physical length to be added to the planar body.

The lengthening section may be considered to be a “second” lengthening section, and the peripheral section may be considered to be a “first” lengthening section, since each section may be shaped to increase a physical length of the elongate track relative to the planar body. The lengthening section may also be referred to as an “excess section” or a “non-linear section”.

The lengthening section may be configured similarly to the lengthening sections discussed in the first aspect. For example, similarly to the lengthening sections discussed in the first aspect, the lengthening section may include one or more deflections (e.g. waves, zig-zags, branches) to cause the elongate track to change direction along the planar body.

The lengthening section may have a similar width to that of the peripheral section. For example, the lengthening section may have a width of less than 40% of a maximum width of the planar body, optionally less than 30%, optionally less than 20%, optionally less than 10%.

Optionally, the lengthening section may be located between a proximal end of the conductive element (e.g. an end which is connected to the inner conductor) and the peripheral section. For example, the elongate track may have an undulating or zig-zag shape in a proximal and/or central region of the instrument tip.

Optionally, the lengthening section may be located between the peripheral section and a terminal end of the conductive element. This arrangement may help ensure that the signal first contacts tissue along the peripheral section, to treat the tissue before additional losses are introduced along the lengthening section.

As used herein, the “terminal end” of the conductive element refers to the end of the conductive element as seen by the working signal when the conductive element is not in contact with tissue. The terminal end may be different from a distal end of the conductive element, since the terminal end optionally may not be located at the distal end of the instrument tip.

As used herein, the phrase “between” may denote that the lengthening section is electrically between two sections (e.g. the peripheral section and the terminal end), as seen by the signal travelling along the electrical element. This may not require the lengthening section to be physically between the peripheral section and the terminal end. For example, the lengthening section and terminal end may each be located physically between two sides of the peripheral section, e.g. by forming a spiral with the peripheral section, with an outer section of the spiral forming the peripheral section and an inward section of the spiral forming the lengthening section and terminal end. The inward section may comprise a linear section (prong) extending longitudinally along the centre of the planar body.

Optionally, the lengthening section may be located in a distal region of the instrument tip. For example, the lengthening section may be located in a distal half of the instrument tip. For example, the lengthening section may be located in a curved tip of the instrument tip. Optionally, the terminal end may also be located at the distal region of the planar body. Locating the lengthening section and/or terminal end at a distal region of the instrument tip can help to provide a desirable energy distribution by directing energy toward the tissue at the distal end of the blade.

Optionally, the peripheral section and lengthening section may together form a spiral at a distal region of the planar body, e.g. as discussed above. A spiral may provide a relatively convenient and compact arrangement for providing a lengthening section which physically sits within the peripheral section. The spiral may be curved or may have sharp edges (e.g. corners).

Preferably, adjacent sections of the spiral (e.g. the peripheral section and additional lengthening section) may be spaced by gaps sized to prevent microwave coupling between the sections. This may help ensure that the working signal travels the full physical length of the conductive track. For example, adjacent sections of the spiral may be separated by gaps having a width of less than 40% of a maximum width of the planar body, optionally less than 30%, optionally less than 20%, optionally less than 10%.

Optionally, the gaps may have the same width as the elongate track. For example, in one embodiment, the elongate track may have a substantially uniform width of approximately 20% of a maximum width of the distal region of the planar body, and the gaps between adjacent sections of the track may also have a substantially uniform width of approximately 20% of the maximum width of the distal region of the planar body. This configuration may allow provide a convenient and equi-spaced spiral arrangement which balances the width of the track and the width of the gaps.

Optionally, the first conductive element is configured to match an impedance of the coaxial feed cable. For example, in a similar manner to the first aspect, the first conductive element may include inductive and/or capacitive elements configured to closely match an impedance of the coaxial feed cable, to help further improve efficiency of energy transfer. For example, the coaxial feed cable may have an impedance of 50 ohms. The conductive and/or inductive elements may be configured in a similar manner to any of the conductive and/or inductive elements as the first aspect, in order to provide the desired impedance at desired dimensions.

The invention of the second aspect may also include other features which may be the same as or similar to those discussed in relation to the first aspect.

For example, optionally, the second conductive element may cover a majority or an entirety of the second surface of the planar body.

Optionally, the instrument tip may include a spacer element proximal to the planar body. The spacer element may be chamfered, and the inner conductor may be electrically connected to the first conductive element by a proximal transmission line which overlies the chamfered spacer element.

Optionally, the instrument may have the same dimensions as those discussed above in relation to the first aspect. Optionally, the instrument tip has a length of 10.0 mm or less. Optionally, the planar body has a width of 1.9 mm or less.

A third aspect of the invention provides an instrument tip having a modified connection to the instrument tip. In particular, the third aspect provides an electrosurgical instrument for applying radiofrequency (RF) electromagnetic (EM) energy and/or microwave frequency EM energy to biological tissue, the instrument comprising: an instrument tip comprising a planar body separating a first conductive element on a first surface thereof from a second conductive element on a second surface thereof, the second surface facing in the opposite direction to the first surface; a coaxial feed cable comprising an inner conductor, an outer conductor coaxial with the inner conductor and a dielectric material separating the inner and outer conductors, the coaxial feed cable being for conveying a working signal comprising an RF signal and/or microwave signal; wherein the inner conductor is electrically connected to the first conductive element and the outer conductor is electrically connected to the second conductive element to enable the instrument tip to receive the working signal, wherein the instrument tip further includes a spacer element proximal to the planar body, wherein the spacer element is chamfered.

Advantageously, the inventors have found that the chamfered spacer element helps to provide a stronger, less brittle connection between the coaxial cable and the planar body of the instrument tip. The chamfered spacer element may also provide a number of additional advantages, for example as discussed elsewhere herein (e.g. in relation to the first aspect).

The coaxial feed cable's outer conductor and/or dielectric material may terminate along the spacer element, so that a distal end of the coaxial feed cable's outer conductor and/or dielectric material are located on the spacer element. The inner conductor of the coaxial cable may protrude beyond the distal end of the coaxial feed cable's outer conductor and/or dielectric material, beyond the spacer element and onto the planar body, to connect with the first conductive element.

The electrosurgical instrument of the third aspect may optionally include any feature(s) discussed above in relation to the first and/or second aspects. For example, the spacer element may optionally include any feature(s) discussed above in relation to the first and/or second aspects. Optionally, the first conductive element may be patterned in any manner discussed in the first or second aspects.

Alternatively, the first conductive element may be patterned in a different manner, to provide different EM properties while retaining the advantages of the chamfered spacer element.

The invention includes the combination of the aspects and preferred features described except where such a combination is clearly impermissible or expressly avoided.

### SUMMARY OF THE FIGURES

Example embodiments illustrating the principles of the invention will now be discussed with reference to the accompanying figures in which like numerals denote like elements.

FIG. 1 is a schematic view of a complete electrosurgery system in which the present invention may be applied.

FIG. 2 is a graph showing variations in electric field and current over the length of a half wave resonator;

FIGS. 3A, 3B, and 3C are a top, side, and bottom views, respectively, of a prior art instrument tip;

FIG. 4 is a top view of an instrument tip according to a first embodiment of the invention;

FIG. 5 is a perspective view of the instrument tip of FIG. 4 connected to a coaxial cable;

FIG. 6 is a top view of an example reference instrument tip for comparison with the first embodiment;

FIG. 7 is a scale drawing showing a side-by-side comparison of the prior art instrument tip and the first embodiment instrument tip;

FIG. 8A is a perspective view of the first embodiment instrument tip with its distal tip inserted into tissue;

FIG. 8B is a top view of the power loss density around the instrument tip in the position of FIG. 8A;

FIG. 8C is a graph of scattering parameters measured in the configuration of FIG. 8A;

FIG. 9A is a perspective view the first instrument tip with only one lateral half of its distal tip inserted into tissue;

FIG. 9B is a graph of scattering parameters measured in the configuration of FIG. 9A;

FIG. 10 is a scale drawing showing a top view of an instrument tip according to a second embodiment of the invention;

FIG. 11 is a perspective view of the instrument tip of FIG. 10

FIG. 12A is a top view of the second embodiment instrument tip with only its distalmost section inserted into tissue;

FIG. 12B is a top view of the power loss density around the instrument tip in the configuration of FIG. 12A;

FIG. 12C is a graph of scattering parameters measured in the configuration of FIG. 12A;

FIG. 13A is a perspective view of the second embodiment instrument tip inserted further into the tissue of FIG. 12A, in a similar position as FIG. 8A;

FIG. 13B is a top view of the power loss density around the instrument tip in the position of FIG. 13A;

FIG. 13C is a graph of scattering parameters measured in the configuration of FIG. 13A;

FIG. 14A is a perspective view of the second embodiment instrument tip with only one lateral half of its distal tip inserted into tissue, in a similar position as FIG. 9A;

FIG. 14B is a top view of the power loss density around the instrument tip in the configuration of FIG. 14A;

FIG. 14C is a graph of scattering parameters measured in the configuration of FIG. 14A;

FIG. 15 is a scale drawing showing a top view of an instrument tip according to a third embodiment of the invention;

FIG. 16 is a perspective view of the third embodiment instrument tip's active tip;

FIG. 17A is a perspective view of the third embodiment instrument tip with only its distalmost section inserted into tissue;

FIG. 17B is a top view of the power loss density around the instrument tip in the configuration of FIG. 17A;

FIG. 17C is a graph of scattering parameters measured in the configuration of FIG. 17A;

FIG. 18A is a perspective view of the third embodiment instrument tip inserted further into the tissue of FIG. 17A, in a similar position as FIGS. 8A and 13A;

FIG. 18B is a top view of the power loss density around the instrument tip in the configuration of FIG. 18A;

FIG. 18C is a graph of scattering parameters measured in the configuration of FIG. 18A;

FIG. 19A is a top view of the third embodiment instrument tip with only one lateral half of its distal tip inserted into tissue, in a similar position as FIGS. 9A and 14A;

FIG. 19B is a graph of scattering parameters measured in the configuration of FIG. 19A;

FIG. 20 is a top view of an instrument tip according to a fourth embodiment of the invention;

FIG. 21 is a scale drawing showing a top view of an instrument tip according to another aspect of the invention;

FIG. 22A is a perspective view showing the instrument tip of FIG. 21 inserted into tissue, in a similar position as FIGS. 8A, 13A, and 18A;

FIG. 22B is a perspective view of the power loss density around the instrument tip in the configuration of FIG. 22A;

FIG. 22C is a graph of scattering parameters measured in the configuration of FIG. 22A;

FIGS. 23A to 23E are top views of the power loss density around the instrument tip of FIG. 21 when inserted at various different positions within tissue;

FIG. 24A is a perspective view showing an embodiment instrument tip with only its distalmost tip inserted into tissue;

FIG. 24B is a graph of scattering parameters measured in the configuration of FIG. 24A;

FIG. 25A is a perspective view showing the instrument tip of FIG. 24A inserted further into tissue;

FIG. 25B is a graph of scattering parameters measured in the configuration of FIG. 25A.

FIG. 26 is a top view of an instrument tip according to an embodiment of the invention; and

FIG. 27 is a graph of scattering parameters measured for the instrument tip of FIG. 26.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

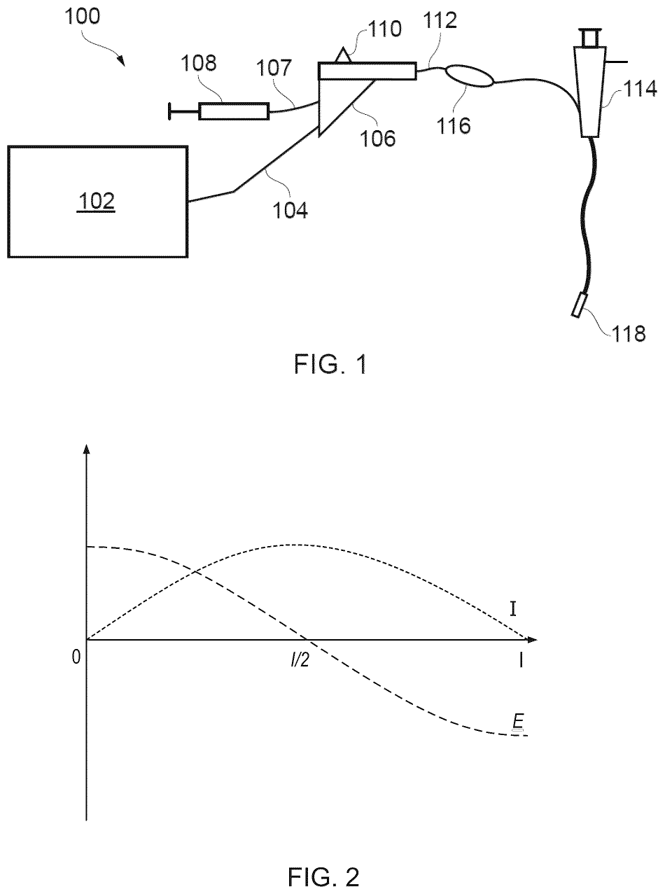

FIG. 1 is a schematic diagram of a complete electrosurgery system 100 that is capable of selectively supplying to the distal end of an invasive electrosurgical instrument any or all of RF energy, microwave energy and fluid, e.g. saline or hyaluronic acid. The system 100 comprises a generator 102 for controllable supplying electromagnetic (EM) energy. In the present embodiment, the EM energy includes RF EM energy and/or microwave frequency EM energy. A suitable generator for this purpose is described in WO 2012/076844, which is incorporated herein by reference.

The generator 102 is connected to an interface joint 106 by an interface cable 104. The interface joint 106 is also connected to receive a pressurised fluid supply from a fluid delivery apparatus 108 via a fluid supply cable 107. The function of the interface joint 106 is to combine the inputs from the generator 102 and fluid delivery device 108 into a single flexible shaft 112, which extends from the distal end of the interface joint 106. It is to be understood that the shaft 112 may form part of the interface joint 106.

The flexible shaft 112 is insertable through the entire length of an instrument (working) channel of a surgical scoping device 114. A torque transfer unit 116 may be mounted on a proximal length of the shaft 112 between the interface joint 106 and surgical scoping device 114. If present, the torque transfer unit 116 engages the shaft to permit it to be rotated within the instrument channel of the surgical scoping device 114.

The flexible shaft 112 has an electrosurgical instrument tip 118 that is shaped to pass through the instrument channel of the surgical scoping device 114 (e.g. an endoscope) and protrude (e.g. inside the patient) at the distal end of the instrument channel. The instrument tip includes an active tip for delivering RF EM energy and/or microwave EM energy into biological tissue and an aperture for delivering pressurised fluid (e.g. saline, Gelofusine, and/or hyaluronic acid with added marker dye). These combined technologies provide a unique solution for cutting and destroying unwanted tissue and the ability to seal blood vessels around the targeted area. By applying pressure to the fluid, the surgeon is able to inject the fluid between tissues layers in order to distend and mark the position of a lesion to be treated. The injection of fluid in this manner lifts and separates the tissue layers making it both easier to resect around the lesion and plane through the submucosal layer, reducing the risk of bowel wall perforation and unnecessary thermal damage to the muscle layer.

The instrument tip 118 may further include a protective hull positioned under the active tip to assist a tissue planing type resection action, again helping to protect against inadvertent perforation and ensure viability of the remaining tissue, which in turn facilitates more rapid healing and post operation recovery.

The structure of the instrument tip 118 may be particularly designed for use with a conventional steerable flexible endoscope having a working channel with an internal diameters of at least 2.2 mm and a working length of between 60 cm and 170 cm. As such the majority of the comparatively small diameter instrument is housed within the lumen of a much larger and predominantly polymer insulating device, i.e. the flexible endoscope channel. In practice, only 5 mm to 25 mm of the distal assembly protrudes from the distal end of the endoscope channel, in order not to block the field of view or adversely affect camera focusing. The protruding part of the distal assembly is the only portion of the instrument that ever makes direct contact with the patient.

At the proximal end of the endoscope working channel, which is typically held 50 cm to 80 cm from the patient, the flexible shaft 112 emerges from the working channel port and extends a further 30 cm to 100 cm to the interface joint 106. In use, the interface joint 106 is typically held by a gloved assistant throughout the procedure. The interface cable 104 is connected to the generator 102 using a QMA-type coaxial interface, which is designed to allow continuous clockwise or counter clockwise rotation. This permits the interface joint 106 to rotate with the torque transfer unit 116 under the control of the user. The assistant supports the interface joint 106 throughout the procedure in order to assist the user with sympathetic instrument rotation and fluid injection.

FIG. 2 shows a graph useful for understanding embodiments of the invention. The graph shows variations in electric field E and current I at various positions along the length l of a half wave resonator. The current I across the length of the resonator follows an approximately sinusoidal half-wave, with a value of 0 at a proximal end of the resonator, a peak in a centre of the resonator, and a value of 0 at a distal end of the resonator. The electric field E follows the corresponding cosinusoidal half-wave, having a peak value at the proximal end of the resonator, a value of zero in the centre of the resonator, and another peak at the distal end of the resonator. The half-waves shown in FIG. 2 will reverse every half-cycle.

In some embodiments, the inventors have developed instrument tips which are configured as half wave resonators. Since inductance is related to current, and since current peaks in the centre of the resonator (as shown in FIG. 2), the inventors have identified that, for a half wave resonator, a central zone of the instrument tip may be considered most important to its inductance. Likewise, since capacitance is related to electric field, and since electric field peaks at the proximal end and distal end, the inventors have identified that proximal and distal zones of the conductive element may be considered most important to the capacitance of the instrument tip.

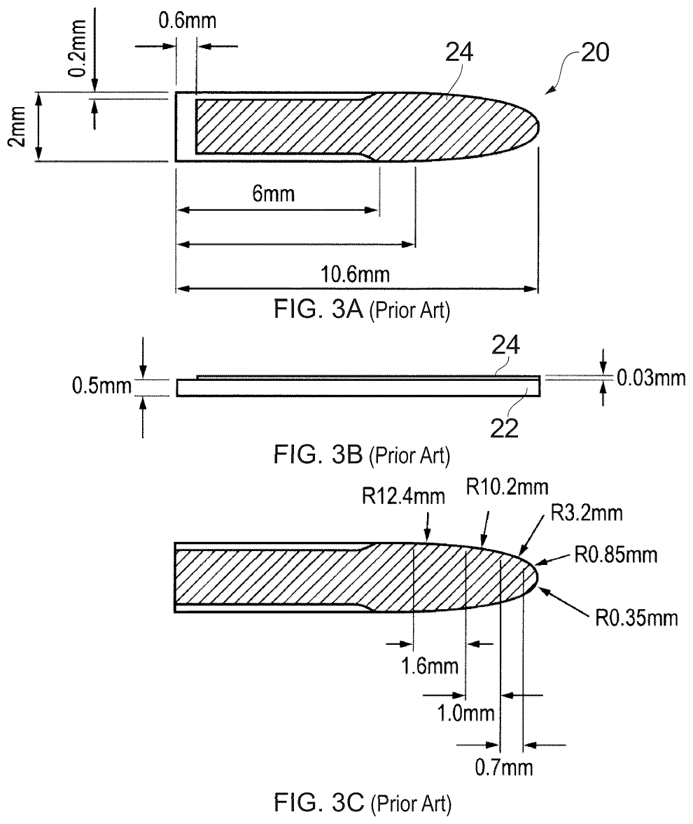

FIGS. 3A, 3B, and 3C illustrate the dimensions of a prior art active tip 20 as discussed in GB 2 523 246. The prior art active tip 20 has a planar body 22 comprising a dielectric substrate separating a first conductive element 24 on a first surface thereof from a second conductive element on a second surface thereof. The overall length of the prior art active tip is 10.6 mm, with a maximum width of 2 mm and a height of 0.5 mm. The first conductive element 24 on the prior art active tip 20 has a thickness of 0.03 mm.

The conductive layers on both surfaces of the planar body 22 are set back from the edges of the planar body 22 by a distance of 0.2 mm along the proximal 6 mm of the tip 20. To ensure that the top conductive layer 24 is isolated from the outer conductor of the coaxial cable, the top conductive layer 24 is set back from the proximal edge of the dielectric substrate by a distance of 0.6 mm.

Because the first conductive element 24 includes a proximal portion which is set back from the edges of the planar body, the first conductive element 24 may be considered to be patterned relative to a reference conductive element that entirely covers a first surface of an equivalent (reference) planar body. However, the set-back pattern in the prior art active tip 20 does not increase the electrical length of the prior art instrument tip compared to an electrical length of the reference instrument tip.

Instead, the set-back conductive area has substantially no effect on the electrical length.

The conductive element 24 includes a patterned segment which is set-back in two zones of the instrument tip: a proximal zone which is generally capacitive in nature, and a central zone which is generally inductive in nature. The set-back patterned segment can be thought of as being half capacitive and half inductive.

In the proximal zone, setting back the conductive element 24 from the edges of the planar body has an effect of reducing the capacitance, since capacitance C is proportional to the overlapping area A of conductive plates as follows:

$$C = \frac{ε A}{d} \qquad ( equation 4 )$$

where ε is dielectric permittivity and d is the distance between the conductive plates.

In the central zone, setting back the conductive element 24 from the edges of the planar body has an effect of increasing the inductance, since narrower transmission lines have higher inductance than wider transmission lines.

Since the conductive element 24 has the same width in the proximal and central zones, i.e. there is no relative difference in widths between the conductive regions in these zones, the reduction in capacitance and increase in inductance effectively cancel out, such that the set-back pattern has substantially no effect on the resonant frequency or the electrical length.

Therefore, as a whole, the set-back pattern in the prior art provides no effect on the electrical length relative to the reference conductive element that covers the entirety of the (equivalent) reference planar body.

It is further noted that the first conductive element 24 includes a curved distal segment. However, the curved distal segment already covers an entirety of the planar body 22 (which is also curved), and therefore its pattern does not provide any change in electrical length relative to a reference conductive element that also covers the entirety of a reference planar body having the same shape and dimensions.

The curvature of the curved distal segment does, however, have an effect of changing the electrical length relative to a rectangular reference conductive element overlying a rectangular reference planar body. However, this also has the effect of decreasing (rather than increasing) the electrical length relative to that of the rectangular conductive element, since it removes capacitance from the distal zone of the instrument. This effect is also explained in GB 2 503 673, which describes the manner in which a similar curved instrument tip needed to be made physically longer in order to provide the required resonant properties, to account for a reduction in capacitance caused by the curved distal tip.

Therefore, in the prior art arrangements, the minimum physical length of the instrument tip was limited by the requirement to provide the necessary electrical length. The instrument tip could not be made smaller, since any reduction in size of the conductive elements would in turn further reduce capacitance C and thus further undesirably increase the electrical length beyond that required for resonance.

In contrast, the inventors have developed modified active tips that can be made smaller than the prior art active tip, whilst maintaining the ability to provide a desired electrical length and efficiently transfer energy into biological tissue.

For example, some embodiments discussed herein provide modifications to the central, proximal, and/or distal zones of an active tip, to help tune its inductance and/or capacitance. In doing so, the inventors have provided active tips which may have reduced sizes compared to the prior art active tip, whilst maintaining functionality at a desired resonant frequency (e.g. 5.8 GHz), and maintaining an efficient impedance match with the target tissue.

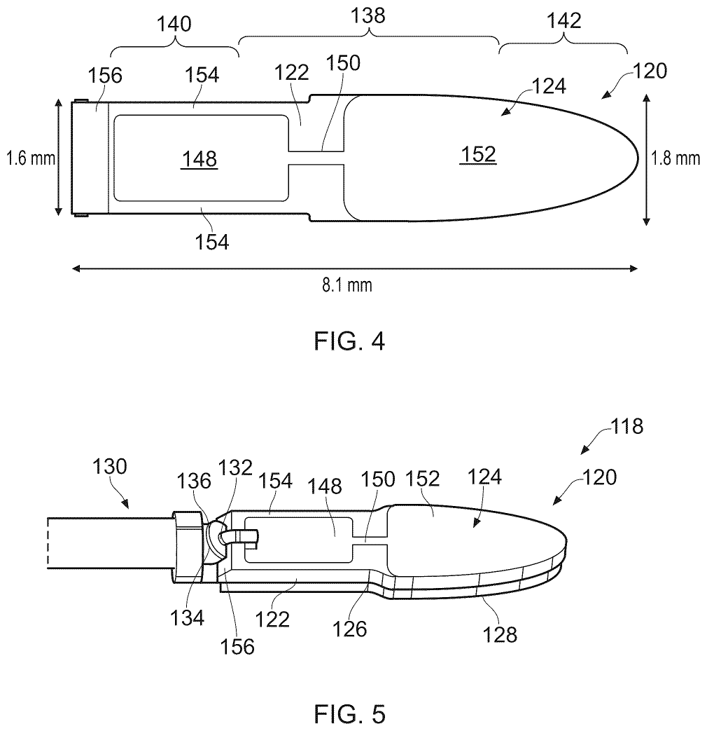

FIGS. 4 and 5 show an instrument tip 118 according to a first embodiment of the invention. The instrument tip 118 includes an active tip 120 having a planar body 122 separating a first conductive element 124 on a first surface of the planar body 122 from a second conductive element 126 on a second surface of the planar body 122. The instrument tip may further include a protective hull 128 on an underside of the active tip 120.

A coaxial feed cable 130 has an inner conductor 132, an outer conductor 134 coaxial with the inner conductor 132, and a dielectric material 136 separating the inner and outer conductors. The inner conductor 132 is electrically connected to the first conductive element 124 and the outer conductor 134 is electrically connected to the second conductive element 126 to enable the active tip 120 to receive the working signal.

The planar body 122 comprises four quarters along its length, the middle two quarters forming a central zone 138 and the proximal and distalmost quarters forming a proximal zone 140 and distal zone 142 respectively.

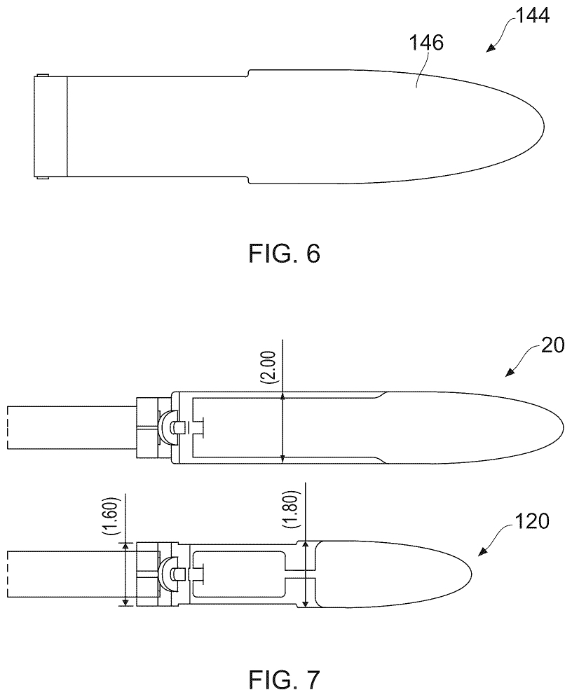

The first conductive element 124 has a pattern configured to set an electrical length of the instrument tip 118 to be greater than an electrical length of a reference instrument tip 144 (which is shown in FIG. 6).

The reference instrument tip 144 is identical to the instrument tip 118 (e.g. having the same shape and physical length) except for the fact that the reference instrument tip 144 includes a reference conductive element 146 that entirely covers the first surface of the planar body of the reference instrument tip 144.

As such, the reference instrument tip 144 is not functional at the same working frequency as the instrument tip 118.

The planar body 122 comprises a dielectric material, for example, alumina having a dielectric constant of 9.8. Under these conditions, a half wave resonator at a frequency of 5.8 GHz would require an electrical electrical length close to

$$\frac{1}{2} λ = \frac{1}{2} \frac{c}{f \sqrt{{ε}_{eff}}} = 8.3 mm .$$

one half wavelength at the frequency of the working signal (e.g. 8.3 mm at a working signal having a frequency of 5.8 GHz).

The first conductive element 124 has a proximal capacitive element 148 in the proximal zone 140, a central inductive element 150 in the central zone 138, and a distal capacitive element 152 in the distal zone 142.

As can be understood with reference to FIG. 2, the proximal zone 140 and distal zone 142 of the instrument tip can each be considered to define areas which contribute predominantly to the capacitance (rather than inductance) of the instrument tip 118. Conversely, the central zone 140 can be considered to define an area which contributes primarily to the inductance (rather than capacitance) of the instrument tip 118.

As can be seen from FIGS. 4 and 5, the proximal capacitive element 148 of the first conductive element 124 is formed as a generally rectangular capacitive plate. The capacitive plate is set-back from the lateral edges of the planar body 122 by a pair of gaps 154. The gaps 154 may help to reduce the transfer of energy from the capacitive element 148 into tissue, thereby helping ensure that energy is primarily transmitted from the distal zone 142 of the instrument tip 118.

The instrument tip further includes a chamfered spacer element 156 proximal to the planar body 122, wherein the inner conductor 132 is electrically connected to the first conductive element 124 by a proximal transmission line which overlies the spacer element 156.

The central inductive element 150 is configured as a straight (linear) thin track connecting the proximal capacitive element 148 to the distal capacitive element 152. In this embodiment, the central inductive element 150 comprises a width that is less than 20% of a maximum width of the instrument tip 118, e.g. approximately 10% of a maximum width of the instrument tip 118.

The distal capacitive element 152 is a continuous metallised layer that extends to the edges of the planar body 122 (i.e. without being set-back by a gap). The distal capacitive element 152 can therefore maintain close contact with tissue to be treated (or diagnosed).

The pattern of the instrument tip 118 differs from a pattern of the reference instrument tip 144 in that the pattern of the instrument tip 118 removes conductive material from the proximal zone 140 (e.g. due to the gaps 154 around the proximal capacitive element 148) and removes conductive material from the central zone (due to the narrow central inductive element 150).

The set-back portions in the proximal zone 140 have an effect of slightly decreasing the electrical length of the instrument tip 118 relative to the electrical length of the reference instrument tip 144. This proximal capacitive section 148 may therefore be considered to provide an electrically shortening pattern. However, the central inductive element 150 is significantly narrowed, providing an effect of increasing its inductance and thereby increasing electrical length to an extent that cancels out and surpasses the effect of any electrical shortening in the proximal zone 140. As a whole, the pattern therefore provides a net effect of setting an electrical length of the instrument tip 118 to be greater than an electrical length of the reference instrument tip 144. For example, in this embodiment, the pattern provides an electrical length of close to 8.3 mm at a working signal having a frequency of 5.8 GHz. The instrument tip 118 is therefore configured as a half wavelength resonator for conveying a working signal comprising a microwave signal at 5.8 GHz. Further, in this embodiment, the active tip 120 achieves this electrical length whilst having a relatively short physical length of 8.1 mm and a maximum width of 1.8 mm.

The instrument tip 118 may therefore be made physically smaller than those of the prior art, and indeed may even be made physically smaller than the calculated electrical length.

In other dimensions, similarly to the prior art active tip 20 of FIGS. 3A to 3C, the active tip 120 may also have a height of 0.5 mm, and the conductive element 124 may have a thickness of 0.03 mm.

In other words, since the conductive element 124 has been made physically smaller than the conductive element 24 of the prior art active tip 20, the active tip 120 will have a relatively lower capacitance than the prior art active tip 20. Accordingly, the conductive element 124 has a modified pattern to provide a relatively higher inductance. This helps to offset the effect of the lowered capacitance, to provide a longer appropriate electrical length. In the present embodiment, this modification includes a central inductive element 150 in the form of a narrow linear track to act as a transmission line connecting the proximal and distal elements 148 and 152. By narrowing the conductive element 124 in the central zone 138, its inductance is increased, and it can therefore be made smaller than the prior art active tip 20 whilst maintaining the required electrical length.

FIG. 7 shows a side-by-side comparison showing the differences in size between the prior art active tip 20 and the modified active tip 120 of the first embodiment. FIG. 7 is drawn to scale, and includes grid spacings of 0.5 mm per square. As can be seen, the modified active tip 120 is significantly shorter than the prior art active tip 20.

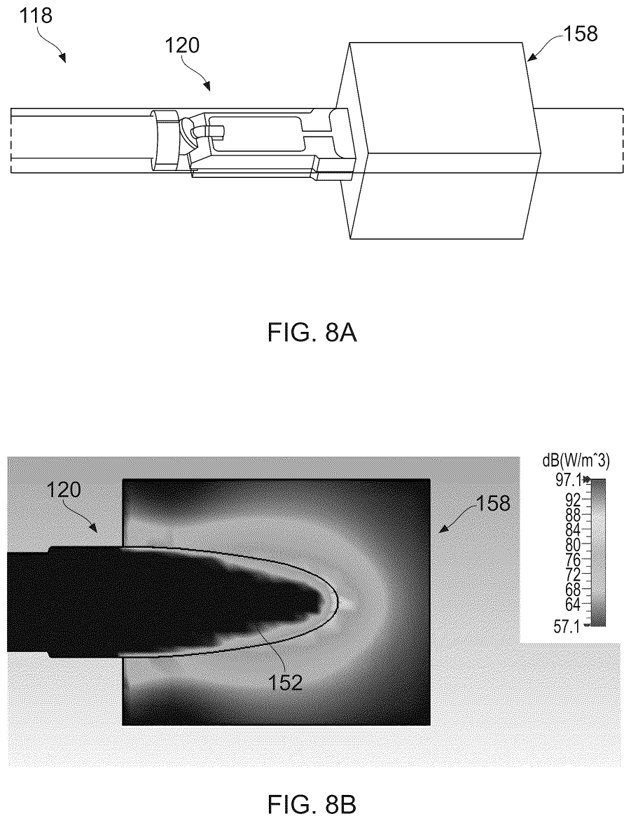

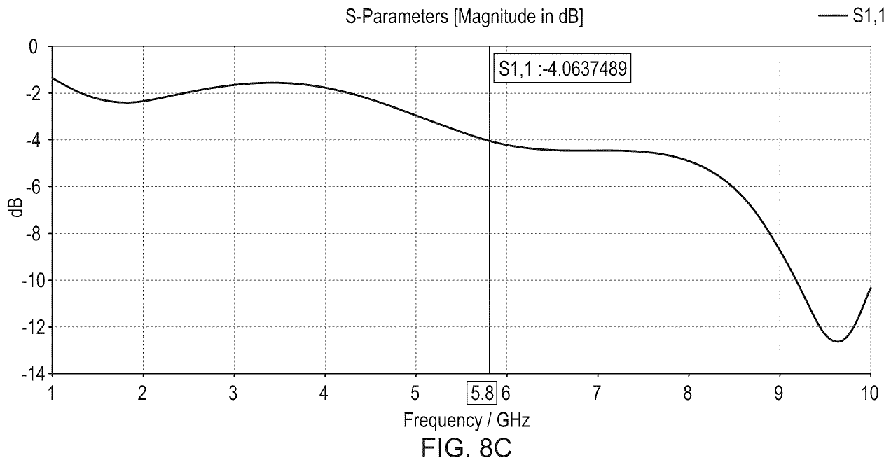

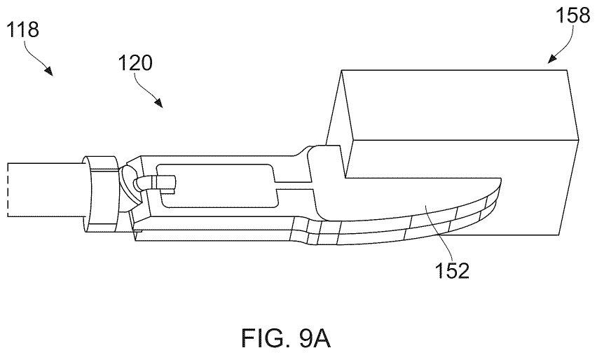

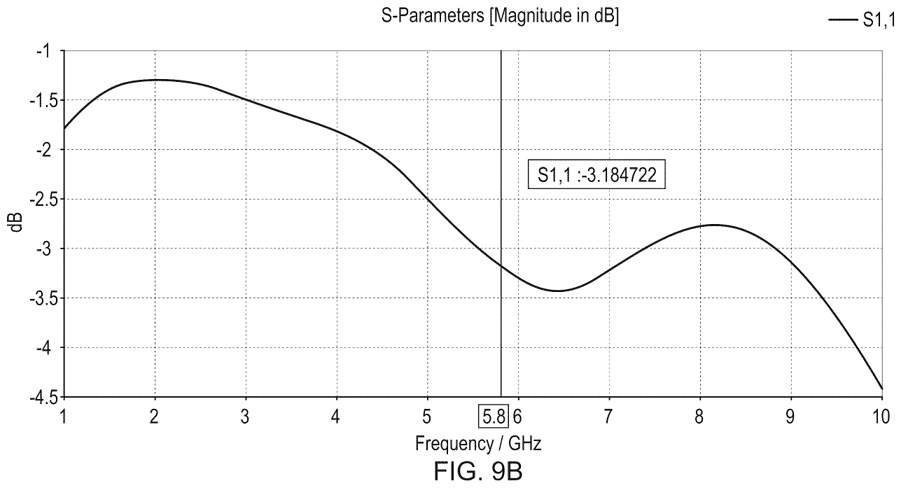

Further, FIGS. 8A to 9B demonstrate that this shorter active tip 120 can still provide reasonably efficient energy transfer into tissue. In particular, FIGS. 8A to 8C and 9A to 9B show the energy distributions of the active tip 120 inserted into liver tissue 158 at two different insertion positions.

FIG. 8A shows the active tip 120 with its distal tip inserted into the tissue 158. FIG. 8B shows the power loss density around the distal capacitive element 152 when inserted into the tissue 158 in the position shown in FIG. 8A. As can be seen from FIG. 8B, the active tip 120 provides a relatively symmetrical and even spread of energy within the tissue 158.

FIG. 8C shows a graph of the scattering parameters (“S-parameters”) provided by the active tip 120 in this position, across various frequencies. This type of graph can effectively provide an understanding of the efficiency with which the active tip 120 transfers energy into the tissue 158, according to the below approximate conversion table:

| | |
|---|---|
| | Percent efficiency |
| dB | (calculated as efficiency = 1 − 10^{dB/10}) |

| | | |
|---|---|---|
| −2 | dB | 37% |
| −3 | dB | 50% |
| −4 | dB | 60% |
| −5 | dB | 68% |
| −6 | dB | 75% |
| −7 | dB | 80% |
| −8 | dB | 84% |
| −9 | dB | 87% |
| −10 | dB | 90% |

As can be seen from FIG. 8C, at a resonant frequency of 5.8 GHz and with the distal tip fully inserted into tissue 158, the modified active tip 120 provides S-parameters of −3.9 dB, so is approximately 60% efficient at transferring energy into the tissue.

As can be seen from FIGS. 9A and 9B, at a resonant frequency of 5.8 GHz and at a second position with the distal tip partially inserted into the tissue 158 (on a lateral side of the distal tip only), the modified active tip 120 provides S-parameters of −3.2 dB, so is approximately 50% efficient at transferring energy into tissue.

The first embodiment is therefore useful at providing relatively consistent efficiency of energy transfer even as the impedance at the distal tip changes (i.e. when changing the amount of tissue in contact with the device). Additionally, the first embodiment provides an even spread of energy around the device (as can be seen from FIG. 8B) whilst providing a relatively small physical arrangement at the desired electric length.

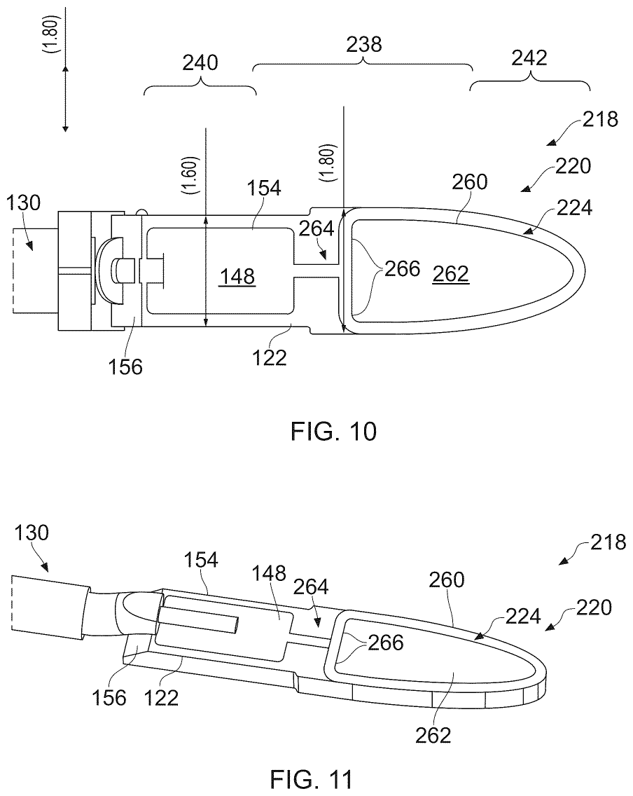

FIGS. 10 and 11 show an active tip 220 according to a second embodiment of the invention. The active tip 220 is generally similar to the active tip 120 of the first embodiment, with similar reference numerals denoting similar elements, except where discussed otherwise. For example, as shown in FIG. 10, the second embodiment may have the same dimensions as the first embodiment. FIG. 10 is drawn to scale and includes grid spacings of 0.5 mm per square.

Compared to the first embodiment, the active tip 220 of the second embodiment may help to further improve the efficiency of energy transfer, by varying the capacitance and inductance of the first conductive element in order to improve the impedance match with the coaxial cable.

In particular, the active tip 220 differs from the active tip 120 in that the active tip 220 has a distal element which has been hollowed out to form a loop 260 around a non-conductive void 262. The loop 260 may therefore be considered to provide both a capacitive effect (at a distal zone 242 of the active tip) and an inductive effect (in a central zone 238 of the active tip 220). In particular, the hollow loop 260 reduces the capacitance of the active tip 220, compared to the active tip 120. Additionally, it effectively provides a lengthened central inductive element 264, e.g. which now further includes a pair of laterally protruding arms 266 in a central zone 238 of the active tip 220, thereby increasing the inductance of the active tip 220. Since impedance is related to capacitance and inductance as

$$Z = \sqrt{\frac{L}{C}} ,$$

this increases the impedance compared to the active tip 220, and can improve its impedance match with the coaxial cable 130.

A curved portion of the conductive loop 260 extends along a curved periphery of the planar body 122 at the distal zone 242, to form a cutting edge for contacting tissue. The loop 260 along the curved periphery of the planar body 122 can act as a convenient indicator for clinicians to more easily visualise the cutting surface of the active tip 220.

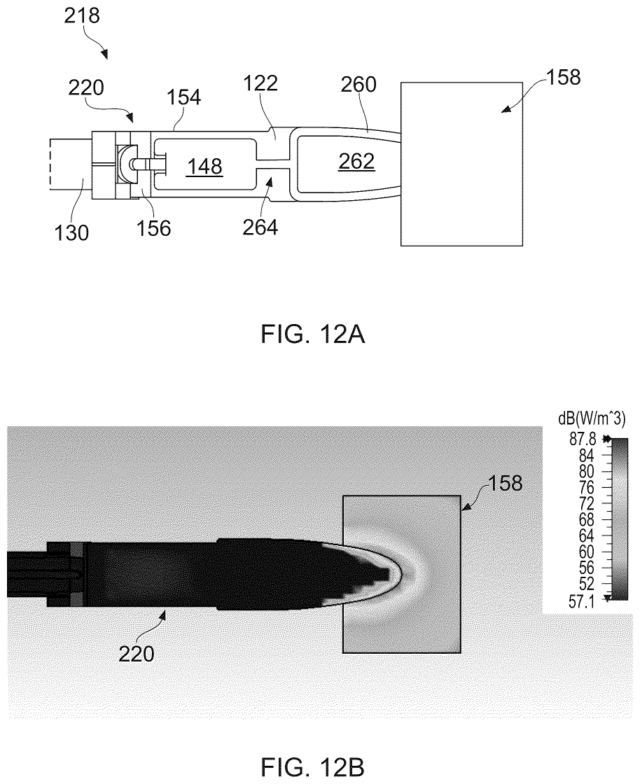

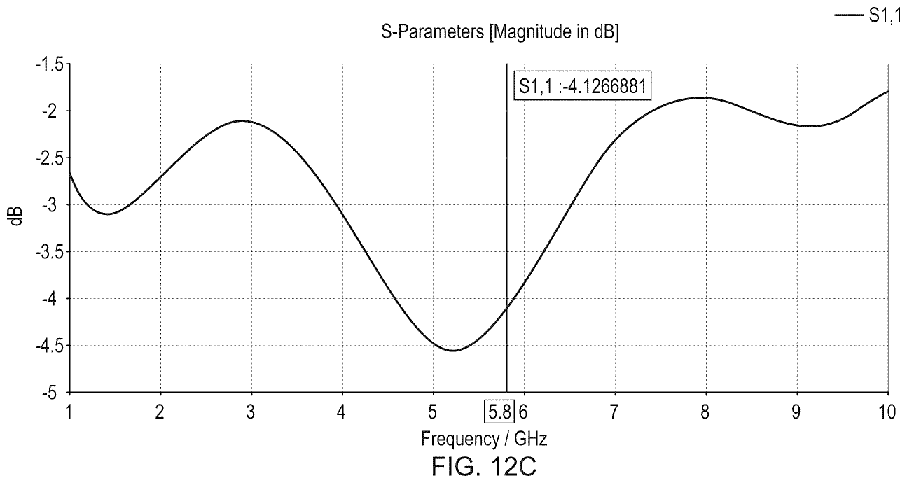

The active tip 220 may advantageously help to provide an even energy distribution and improve the efficiency of energy transfer, even when varying the position of the active tip 220 with respect to tissue. For example, FIGS. 12A to 14C show the efficiency and energy distributions of the active tip 220 at three different positions with respect to the liver tissue 158.

FIG. 12A shows the active tip 220 at a first position, with only a distalmost portion of the active tip 220 inserted into the tissue 158. As shown in FIG. 12B, this provides a relatively even energy distribution around the active tip. As shown in FIG. 12C, the resulting S-parameter at 5.8 GHz is −4.1 dB, indicating that the active tip 220 is approximately 60% efficient at this frequency and position.

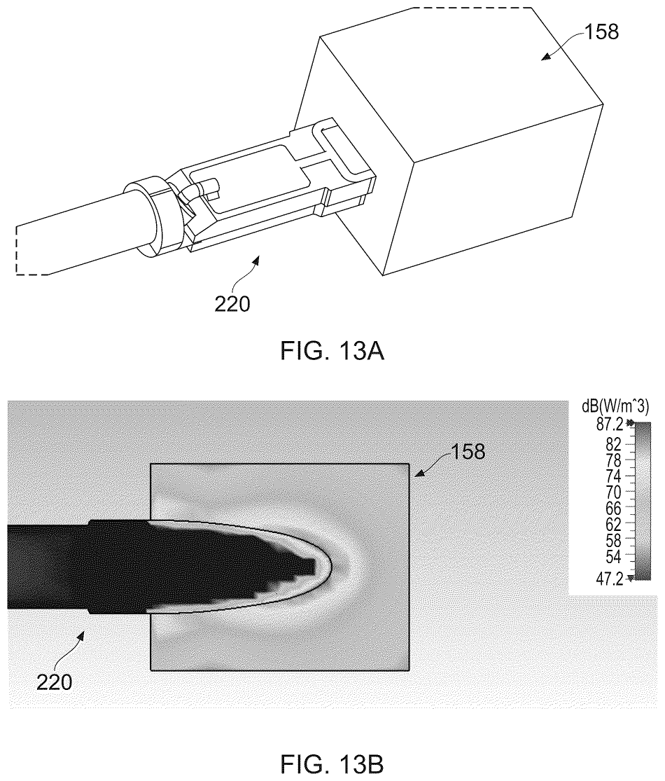

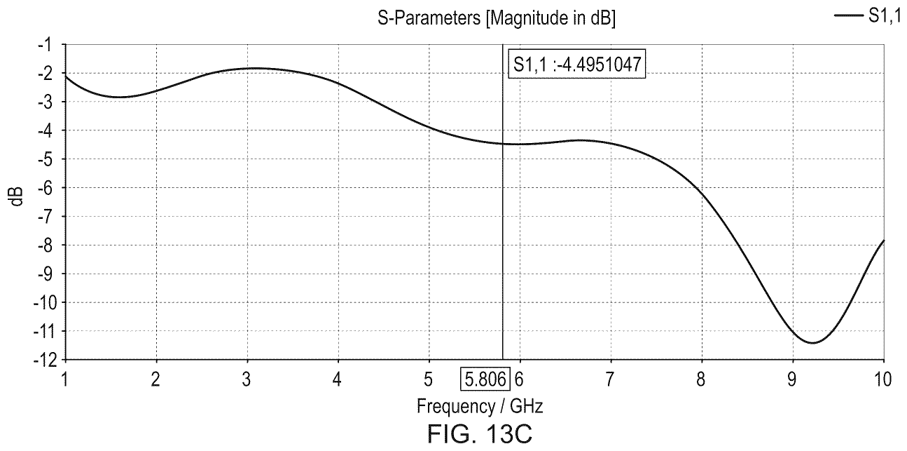

FIG. 13A shows the active tip 220 at a second position, in which it is further inserted into the tissue at a greater depth than the first position. As shown in FIG. 13B, this also provides a relatively even spread of energy around the active tip 220. Further, as shown in FIG. 13C, the resulting S-parameter at 5.8 GHz is −4.5 dB, indicating that the active tip 220 is approximately 65% efficient at this frequency and position.

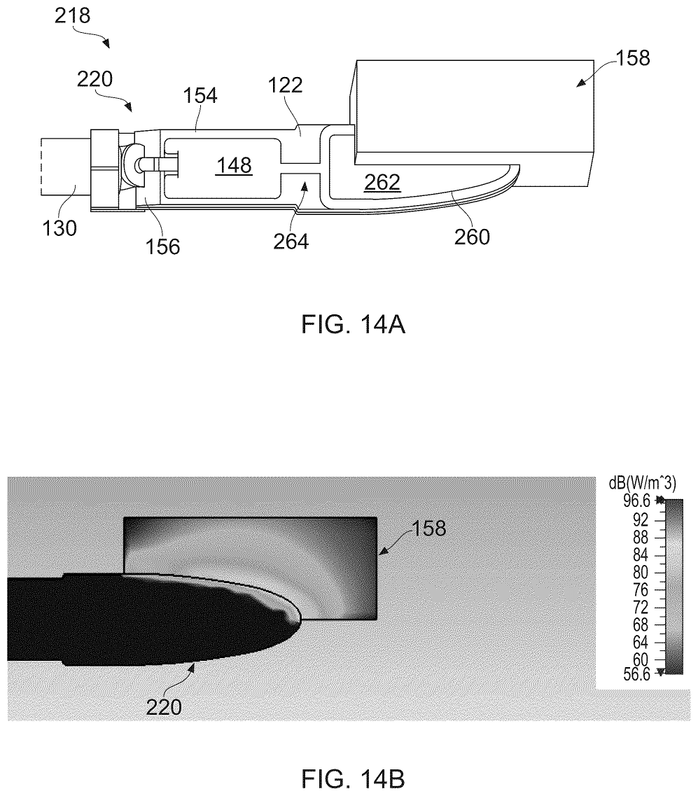

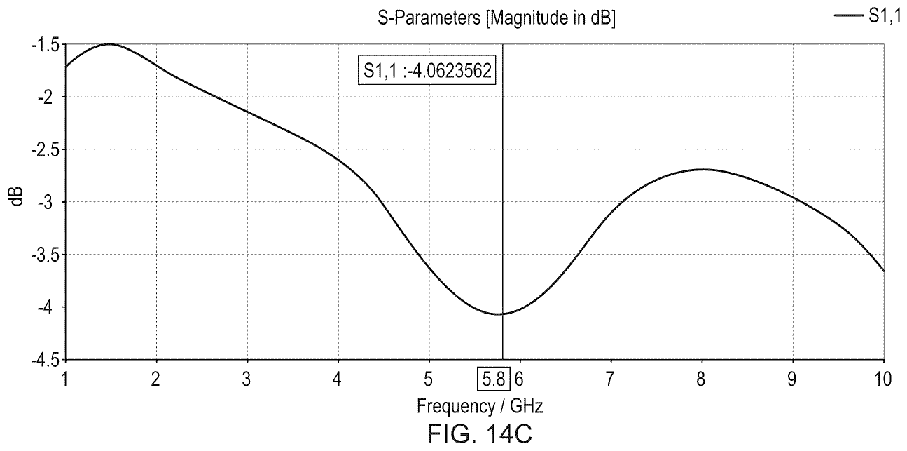

FIG. 14A shows the active tip 220 at a third position. In this position, the active tip 220 is inserted into the tissue 158 at the same depth as the second position, but only along one lateral side of the active tip 220. As shown in FIG. 14B, this also provides a relatively even spread of energy around the active tip. Further, as shown in FIG. 14C, the resulting S-parameter is −4.1 dB at 5.8 GHz, indicating that the active tip 220 is approximately 60% efficient at this frequency and position.

The active tip 220 is therefore useful at improving the efficiency of energy transfer compared to the active tip 220, and improving the consistency of that efficiency even as the impedance at the active tip 220 changes (i.e. when changing the amount of tissue in contact with the device).

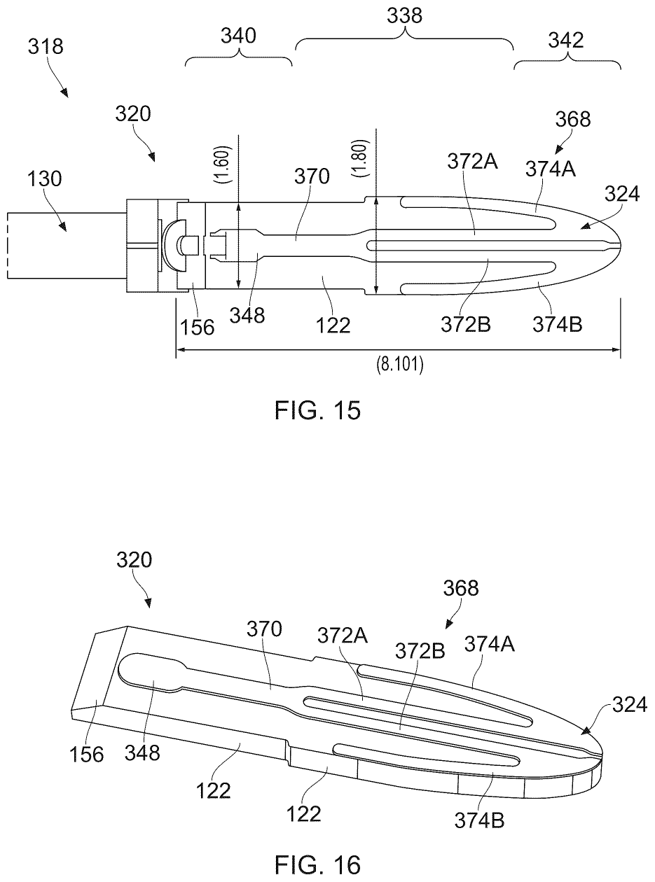

FIGS. 15 and 16 show an active tip 320 according to a third embodiment of the invention.

The figures showing active tip 320 include similar reference numerals to the figure showing active tips 120 and 220 to denote similar elements, except where discussed otherwise. The active tip 320 has the same dimensions as the active tips 120 and 220. FIG. 15 is drawn to scale and includes grid spacings of 0.5 mm per square.

The active tip 320 differs from the active tips 120 and 220 in that it has a narrowed proximal section which further reduces the capacitance of the conductive track (which in turn reduces electrical length). To compensate for this and electrically lengthen the active tip 320 to the required length, the active tip 320 includes a pair of lengthening sections 368 configured to increase the physical length of the conductive element 324 relative to a physical length of the planar body 122.

More specifically, at a proximal zone 340 of the active tip 320, the first conductive element 324 includes a capacitive element 348 comprising a plate (or “bulb”) which is significantly narrower than the planar body 122 (e.g. approximately ⅓ of the width of the proximal zone 340 of the planar body 122). The conductive element 122 further includes a conductive track 370 longitudinally extending from the capacitive element 348 through a distalmost section of the central zone 342. At approximately the centre of the planar body 122 (i.e. where the body begins to curve inwardly), the conductive track 370 forks into a pair of tracks 372A and 372B which are spaced from each other and extend to the distal tip of the planar body 122. At the distal tip, the tracks 372A and 372B then extend outwardly along respective peripheral edges of the planar body 122 to form a pair of branches 374A and 374B projecting backwards towards a proximal region of the planar body 122. In this embodiment, each branch 374A and 374B ends in a respective terminal end located at a respective peripheral edge of the central zone 338 of the active tip 320. Under this arrangement, the effective centre of the capacitance at the distal end is approximately halfway along the branches 374A and 374B on the periphery of the planar body 122.

The branches 374A and 374B effectively serve to increase the physical length of the conductive element 324, in turn increasing its electrical length as seen by a working signal conveyed along the active tip 320. Since the branches 374A and 374B extend along the periphery of the planar body 122, they are also used to perform a working (treatment or diagnostic) function and may provide an easily visualised working surface for a clinician operating the instrument.

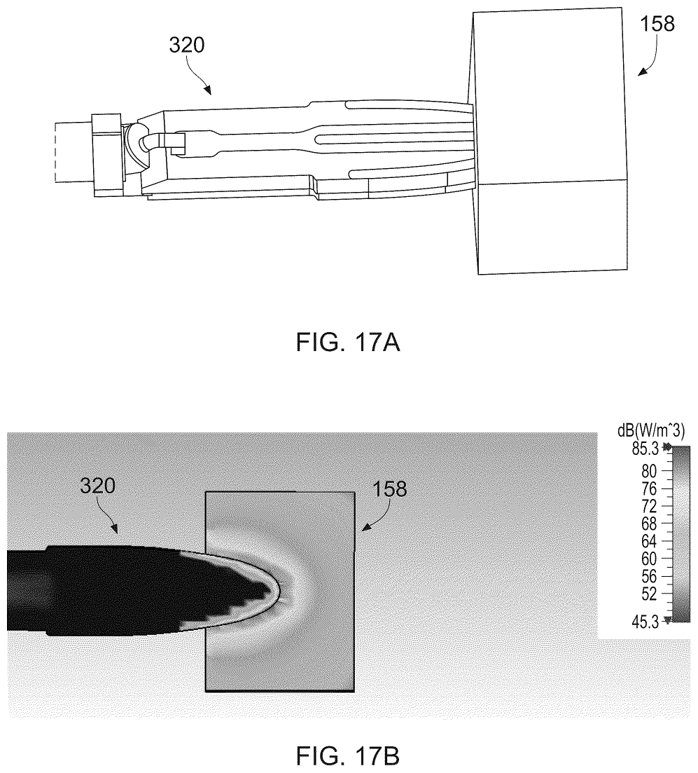

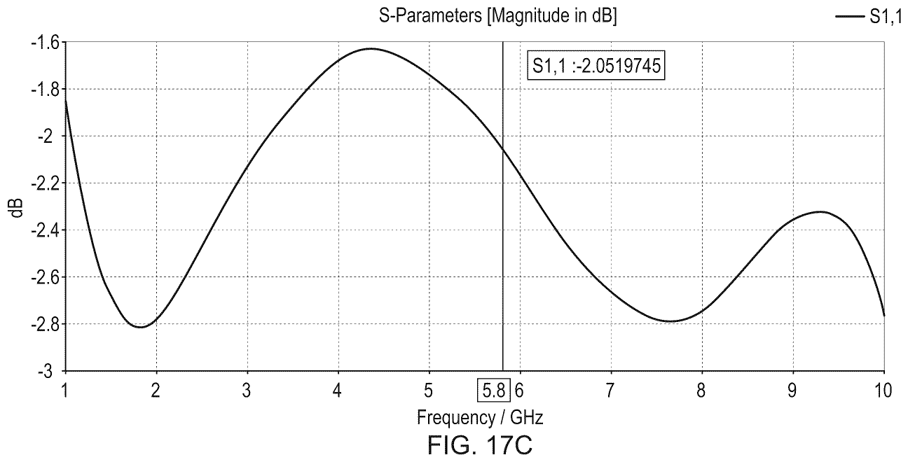

FIGS. 17A to 19B show the efficiency and energy distributions of the active tip 320 at three different positions with respect to the liver tissue 158.

FIG. 17A shows the active tip 320 at a first position, with only a distalmost portion of the active tip 320 inserted into the tissue 158. As shown in FIG. 17B, this provides a relatively even energy distribution around the active tip. As shown in FIG. 17C, the resulting S-parameter at 5.8 GHz is −2.05 dB, indicating that the active tip 320 is approximately 37% efficient at this frequency and position.

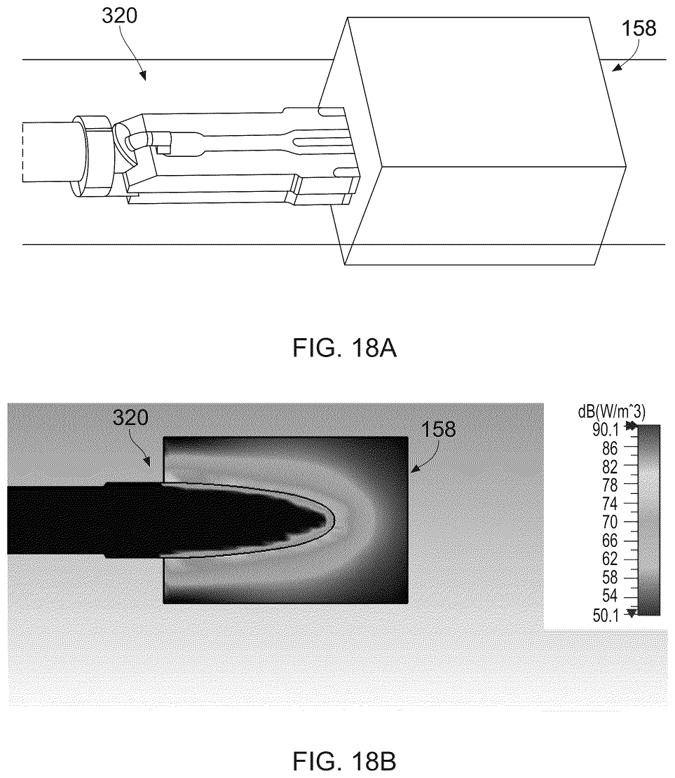

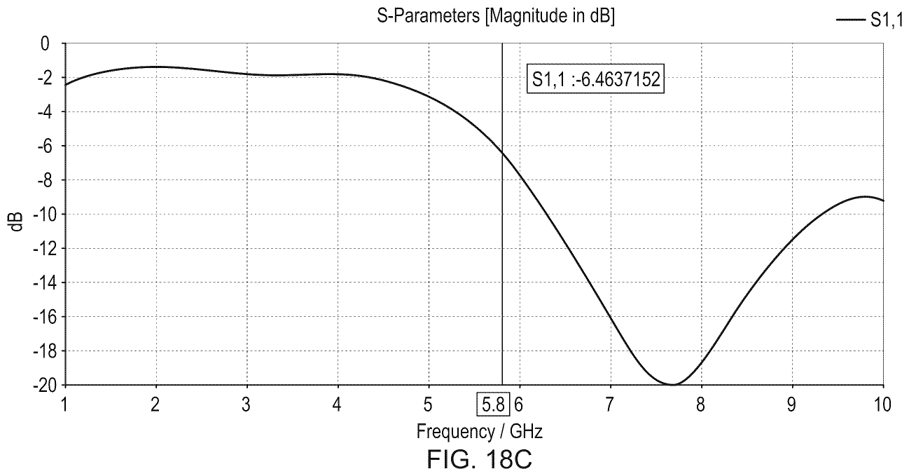

FIG. 18A shows the active tip 320 at a second position, in which it is further inserted into the tissue 158 at a greater depth than the first position. As shown in FIG. 18B, this also provides a relatively even spread of energy around the active tip 320. Further, as shown in FIG. 18C, the resulting S-parameter at 5.8 GHz is −6.5 dB, indicating that the active tip 320 is approximately 78% efficient at this frequency and position.

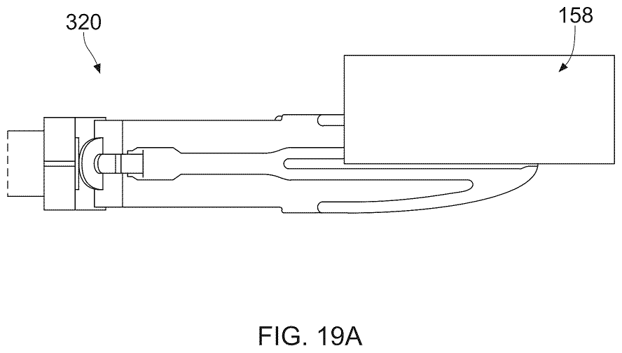

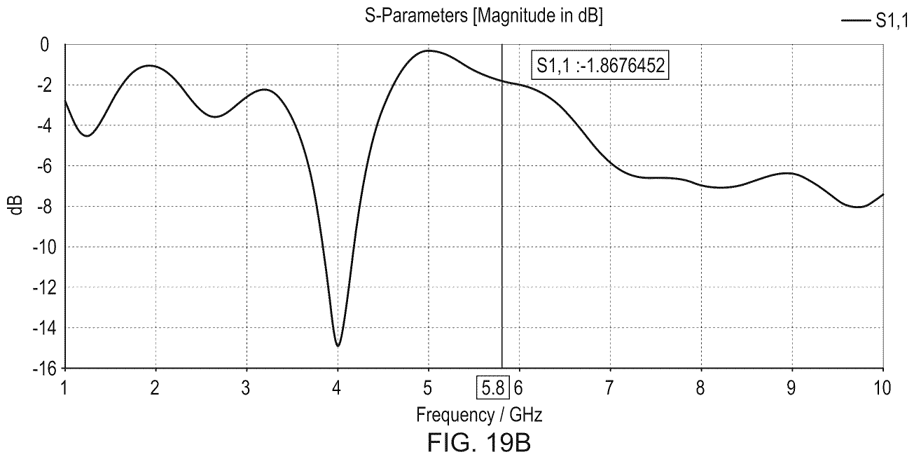

FIG. 19A shows the active tip 320 at a third position. In this position, the active tip 320 is inserted into the tissue 158 at the same depth as the second position, but only along one lateral side of the active tip 320. As shown in FIG. 19B, this provides a resulting S-parameter of −1.9 dB at 5.8 GHz, indicating that the active tip is approximately 35% efficient at this frequency and position.

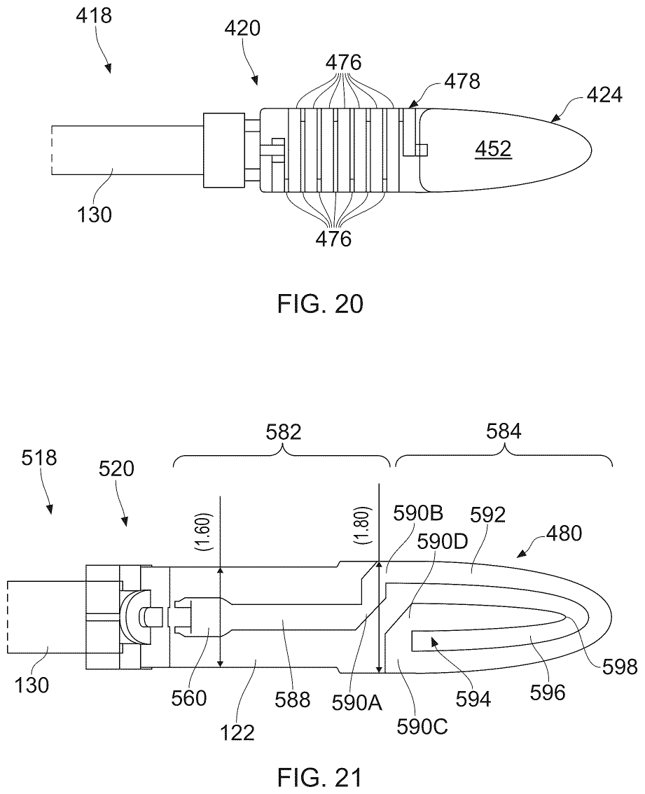

FIG. 20 shows another instrument tip 418 that includes an active tip 420 having a first conductive element 424 that aims to increase its physical length relative to the physical length of the planar body.

In a distal portion of the active tip 420 (e.g. a distal 40% of the active tip), the conductive element 424 extends across the entirety of the planar body, to form a distal capacitive element (capacitive plate) 452.

In a proximal portion of the active tip (e.g. a proximal 60% of the active tip), the conductive element 424 is patterned as an elongate track having a plurality of deflections 476 along its length to form a square-wave shape 478 extending between the coaxial cable 130 and the distal capacitive element 452.

This square-wave track was designed to increase the physical length of the conductive element relative to the physical length of the planar body and in turn increase the electrical length of the conductive element relative to a reference conductive element extending over the entirety of the surface of the planar body.

However, if adjacent sections of the square-wave track are very close to each other, the working signal (e.g. microwave frequency energy at 5.8 GHz) may couple across the square waves in use, effectively bypassing the square wave portions and travelling directly to the distal end (e.g. along a path roughly aligned with a longitudinal axis of the instrument tip).

Therefore, according to this embodiment, this pattern is selected to size the gaps between adjacent conductive sections (e.g. to be 1 mm from each other, for a planar body having a thickness of 0.5 mm), so as to prevent the working signal from coupling across the gaps. This arrangement provides a lengthened section which is configured to convey the working signal around a lengthened portion of track, to thereby increase its electrical length.

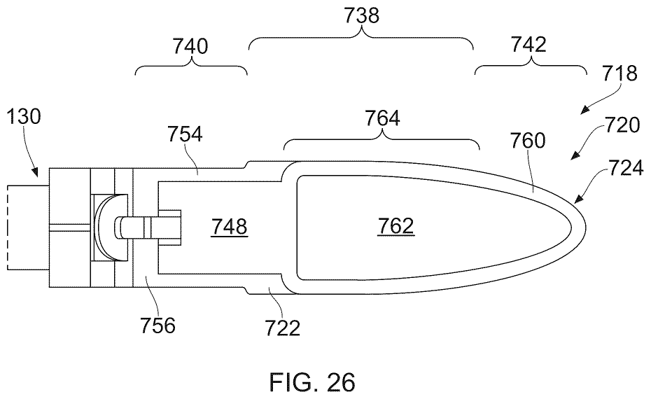

FIG. 26 shows an electrosurgical instrument tip 718 having an active tip 720 according to another embodiment of the invention. FIG. 26 includes similar reference numerals to those discussed above to denote similar elements, except where discussed otherwise.

The instrument tip 718 is similar to the instrument tip 218 of FIGS. 10 and 11, but has a different pattern that allows the instrument tip 718 to be made even smaller whilst still providing an appropriate electrical length and impedance match. In particular, the instrument tip 718 has a pattern that does not include the central longitudinally extending portion of the inductive element 264. Instead, the instrument tip 718 has a conductive loop 760 around a non-conductive void 762 that connects directly to a distal end of a conductive (capacitive) plate 748 and that provides the desired inductance.

In more detail, as shown in FIG. 26, the active tip 718 may be significantly shorter than the active tip 218. In this embodiment, the active tip 718 has a physical length of 6.56 mm and is configured to operate as a half wave resonator at a working frequency of 5.8 GHz.

Compared to the embodiments discussed above, the active tip 718 is shortened in a proximal (rectangular) portion of its planar body 722, i.e. the active tip 718 may have a distal portion (e.g. curved portion) that has the same length as the curved distal portion in FIG. 10 and may therefore provide a similar interaction with tissue, e.g. by providing a similarly sized cutting surface.

The active tip 718 includes a proximal zone 740 in a proximal quarter of the planar body 722, a central zone 738 in the central two quarters of the planar body 722, and a distal zone 742 in a distal quarter of the planar body 722.

The active tip 718 includes a conductive element 724 that is patterned in the proximal zone 740 to include a rectangular capacitive element 748. The capacitive element 748 is generally similar to the capacitive elements 148 and 248 discussed in relation to FIGS. 4 and 10.

At a distal end of the capacitive element 748 (which lies within the central zone 738), the capacitive element 748 is connected directly to the conductive loop 760. A portion of the conductive loop which lies in the central zone 738 may be considered to act as a pair of inductive elements that extend around opposing peripheries of the central zone 738 of the planar body 722, in a longitudinal direction toward a distal end of the active tip 720. This arrangement can increase the inductance in the central zone 738 compared to the pattern shown in FIG. 10, and therefore allows the size of the instrument tip to be even further reduced whilst maintaining the desired electrical performance.

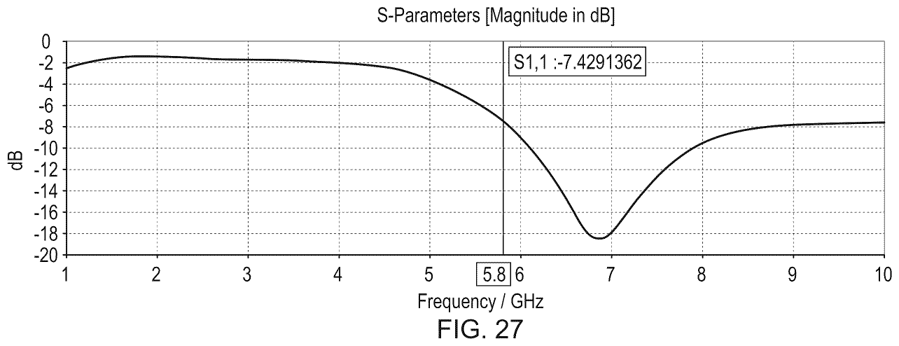

For example, FIG. 27 shows the S-parameter for the active tip 720 inserted into liver tissue 158. As shown in FIG. 26, the active tip 720 provided an S-parameter of −7.4, indicating an efficiency of approximately 82% at this frequency. The active tip 720 was also found to have high efficiencies and an even distribution of energy when changing its position relative to the liver 158.

FIGS. 21 to 23E show an electrosurgical instrument tip 518 having an active tip 520 according to the second aspect of the invention. FIGS. 21 to 23E include similar reference numerals to those discussed above to denote similar elements, except where discussed otherwise. FIG. 21 is drawn to scale and includes grid spacings of 0.5 mm per square. The active tip 520 may have the same dimensions as the active tips 120, 220, 320, and 420.

The active tip 520 may even further improve the efficiency of energy transfer, compared to the active tips 120, 220, 320, 420, and/or compared to active tips discussed in the prior art.

In contrast to the previous active tips (which are each configured as half wave resonators), the active tip 520 is not configured as a resonator. Instead, the active tip 520 is configured as a lossy transmission line.

The active tip 520 includes an elongate track 580 that extends from a proximal region 582 of the planar body 122, around a periphery of a distal region 584 of the planar body 122 for contacting tissue.

In the proximal region (e.g. proximal half 582 of the active tip 520, the elongate track 580 includes an enlarged proximal end 586 for connecting to the coaxial cable 130. The elongate track 580 further includes a narrower thin track, e.g. having a straight (linear) section 588 which extends longitudinally along a central axis of the planar body. The size of the proximal end 586 and/or straight section 588 may be selectively configured to provide an impedance match with the coaxial cable 130 and/or tissue. For example, the enlarged proximal end 586 may have a width of approximately 35% of the width of the proximal region 582 of the planar body 122, and the straight section 588 may have a narrower width of approximately 25% of the width of the proximal region 582 of the planar body 122. In variant embodiments, the thin track might not be straight, e.g. it could have an undulating shape or other non-linear shape.

At an end of the straight section 588, the elongate track 580 includes a first deflection 590A (bend, corner) and changes direction to extend transverse to the longitudinal axis of the planar body, i.e. toward a peripheral edge of a distal region 584 (e.g. distal half) of the active tip 520. The elongate track 580 then further includes a second deflection 590B (bend, corner) to change direction to extend around the periphery of the distal region 584. The section of the elongate track 580 which extends around the periphery (along the edges) of the distal region 584 of the planar body 122 may be referred to as a peripheral section 592.

The peripheral section 592 ends with a third deflection 590C (corner) at an opposite side of the planar body 122 from the second deflection 590B.

After the third deflection 590C, the elongate track 580 includes a lengthening section 594 comprising a fourth deflection 590D (corner) that turns to a linear track (prong) extending longitudinally toward the distal end of the active tip 520. The peripheral section 592 and lengthening section 594 therefore together form a spiral at the distal region 584 of the active tip 520, the peripheral section 592 defining an outer portion of the spiral and the lengthening section 594 defining an inner portion of the spiral.

The peripheral section 592 and lengthening section 594 form adjacent portions of the spiral that are separated along their lengths by a U-shaped gap 596. The gap 596, peripheral section 592, and lengthening section 594 each have a width of approximately 20% of a maximum width of the distal region (although their widths may vary and taper slightly e.g. toward the narrower distal end of the active tip 520). The relatively large gap 596 may help to prevent the working signal from coupling across adjacent sections of the spiral.

In use, the active tip 520 can be brought into contact with tissue, which acts as a load at the distal end of the active tip 520. The active tip 520 can convey the working signal along the straight section 588, and around the peripheral section 592. Since the peripheral section 592 is relatively narrow (e.g. less than 0.5 mm in width), it can provide a relatively high energy distribution across its width, in close contact with the tissue. Further, since the peripheral section 592 is relatively long (e.g. extending around a distal 40% of the planar body), it can transfer this energy into tissue along a longer length of track (e.g. compared to a conductive element that covers the entire planar body). The working signal may therefore be efficiently conveyed into tissue, and in turn may substantially decrease in amplitude along the elongate track 580, so as to provide a significantly reduced amplitude at a terminal end 598 of the elongate track 580.

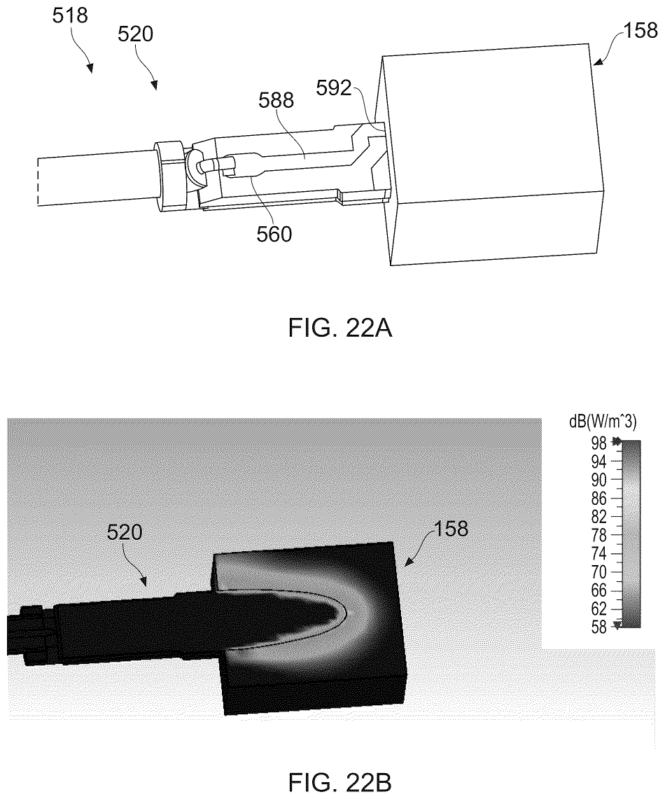

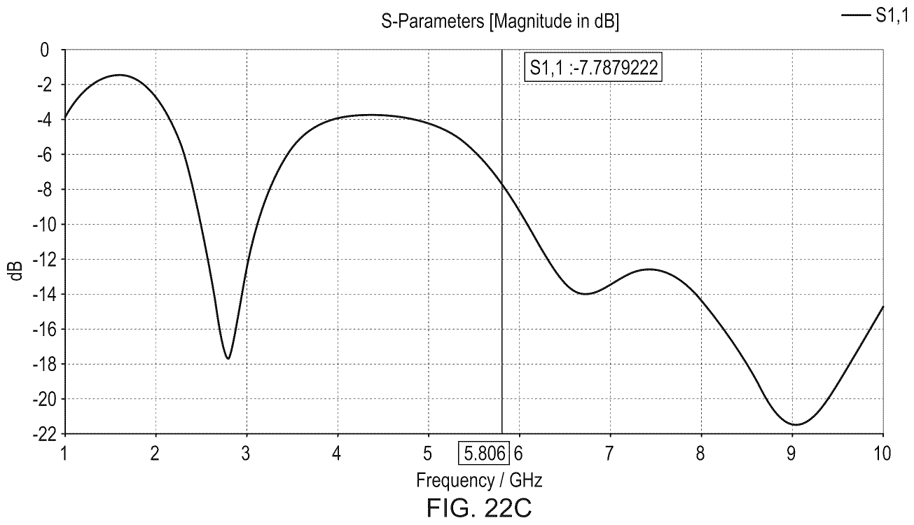

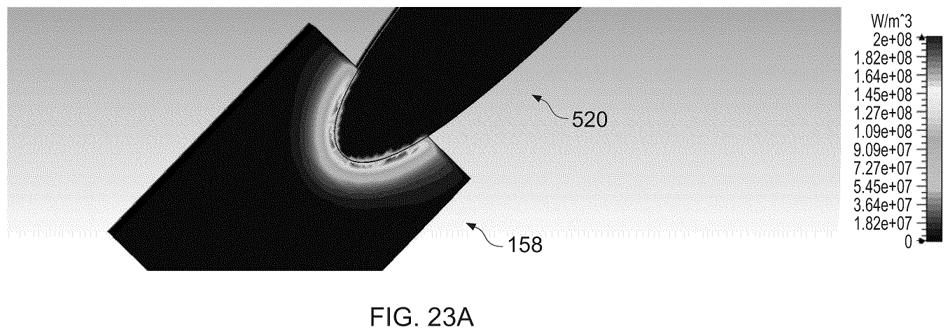

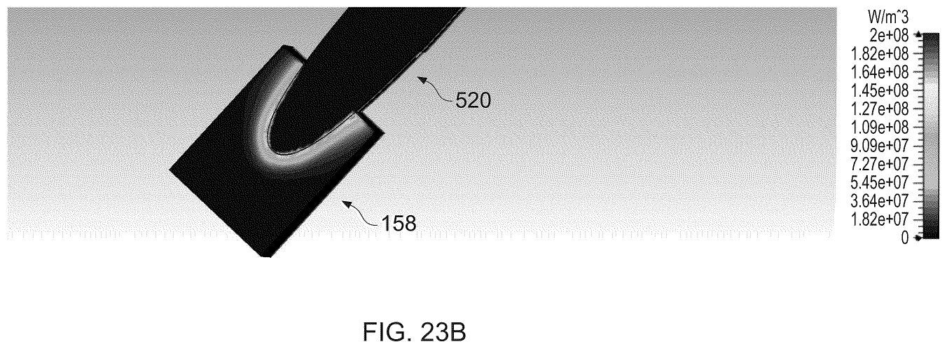

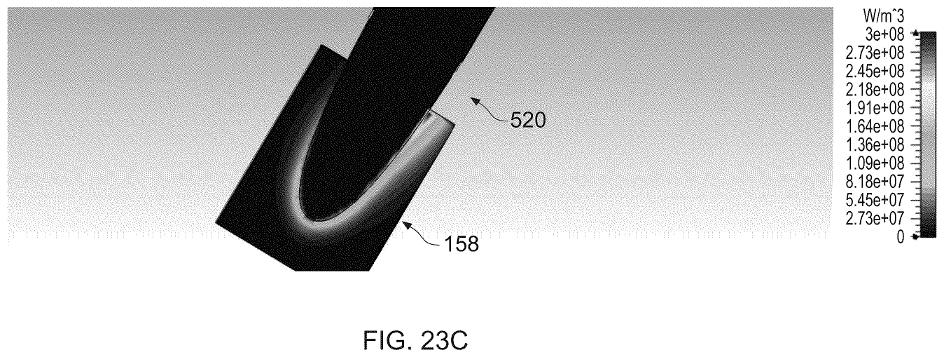

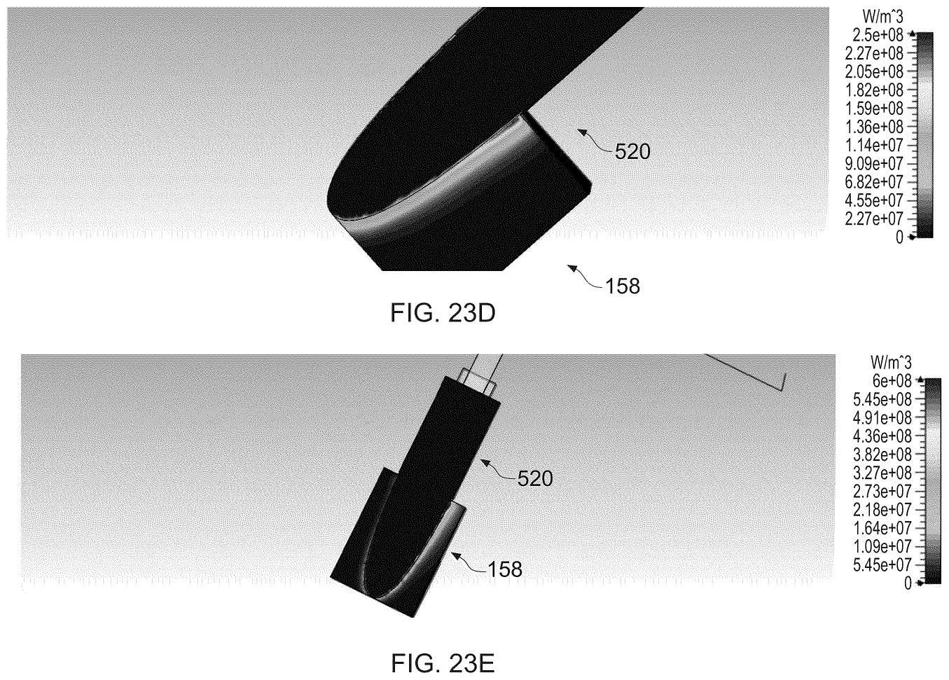

FIGS. 22A to 23 show the efficiency and energy distributions of the active tip 520 at two different positions with respect to the liver tissue 158.

FIGS. 22A to 22B show the active tip 520 in which the majority of the peripheral section 592 is inserted into tissue. As shown in FIG. 22B, this provides a larger amount of energy into tissue near an input end of the peripheral section 592 (i.e. electrically nearer the coaxial cable 130) than near an output end of the peripheral section 592 (i.e. electrically nearer the lengthening section 594).

Similarly, FIGS. 23A to 23E show the energy distribution at various positions as the active tip 520 is inserted into the tissue 158. As shown in these figures, the power density is greatest near the input end of the peripheral section 592, and tapers around the length of the peripheral section 592. The power density also tapers in the same manner if the tissue (load) is only on one lateral side of the active tip, as shown in FIG. 23D. In variant embodiments, the width of the peripheral track and/or its closeness to the edge of the planar body could be modified to provide a more uniform power absorption.

The active tips 120, 220, 320, and 420 discussed above may therefore provide a more even energy distribution than the active tip 520, since the energy distribution is symmetrical on both lateral sides of the active tips 120, 220, 320, and 420. However, the active tip 520 may provide an arrangement that can more efficiently transfer energy into tissue. For example, as shown in FIG. 22C, the resulting S-parameter in the position of FIGS. 22A and 22B at 5.8 GHz is −7.8 dB, indicating that the active tip 520 is approximately 83% efficient at this frequency and position.

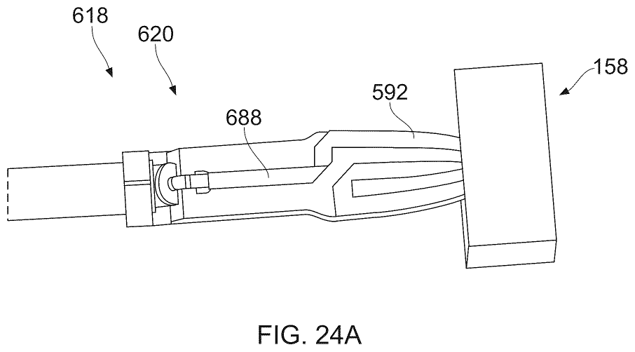

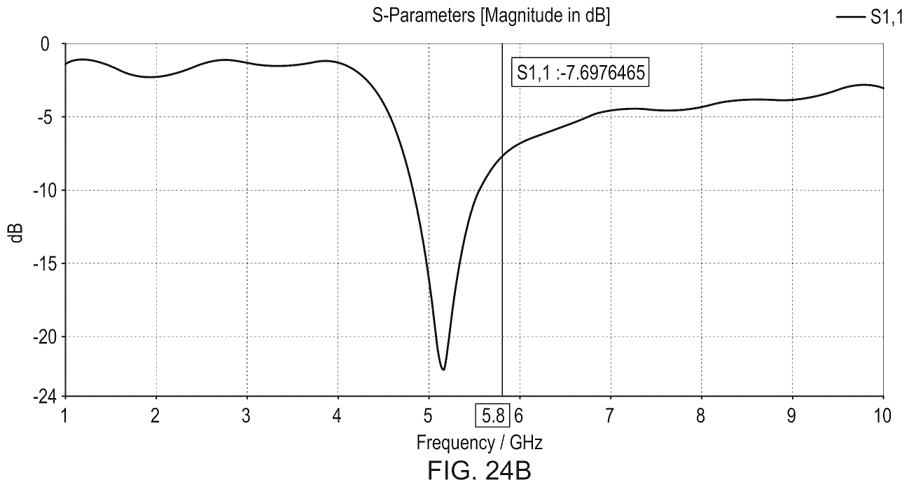

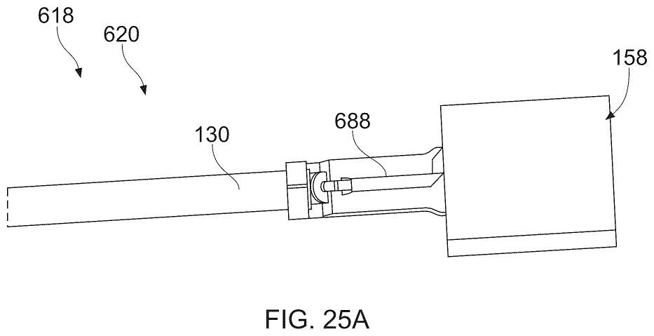

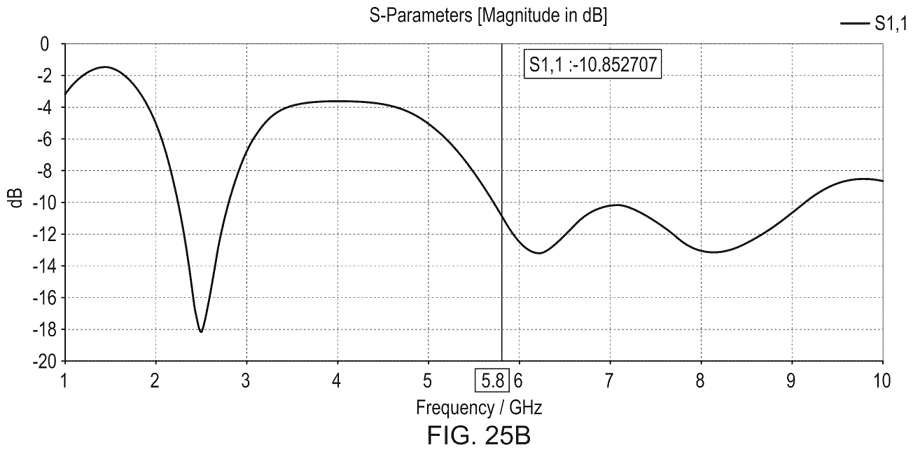

FIGS. 24A to 25B show an electrosurgical instrument tip 618 having an active tip 620 according to another embodiment of the second aspect of the invention.

The active tip 620 is similar to the active tip 520, but does not include a proximal end that is enlarged relative to the straight track in the proximal region. Instead, the conductive element 624 has a straight track 688 which extends to the proximal end and has a constant width along its entire length.

FIG. 24B shows the S-parameter for this active tip 620 at the position of FIG. 24A (in which the liver tissue 158 covers only a tip of the peripheral section 620). This provides an S-parameter of −7.7, indicating that the active tip 620 has 83% efficiency at this frequency and position. FIG. 25B shows the S-parameter for the active tip 620 at a position of FIG. 25B (in which the liver tissue covers the entirety of the peripheral section 592). This provides an S-parameter of −10.8, indicating that the active tip 620 has 92% efficiency at this frequency and position.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%,
100 electrosurgery system
   102 generator
   104 interface cable
   106 interface joint
   112 flexible shaft
      118 electrosurgical instrument tip
   107 fluid supply cable
   114 surgical scoping device
   116 torque transfer unit
   108 fluid delivery apparatus
   20 Prior art active tip
      22 planar body
      24 first conductive element
   118 instrument tip
      120 active tip
         138 central zone
         140 proximal zone
         142 distal zone
         122 Planar body
         124 First conductive element
         148 proximal capacitive element
         150 central inductive element
         152 distal capacitive element
         126 Second conductive element
         154 gaps
         156 spacer element
      128 Protective hull
      130 coaxial feed cable
         132 inner conductor
         134 outer conductor
         136 dielectric material
   144 reference instrument tip
      146 reference first conductive element
   158 Tissue sample
   218 instrument tip
      220 active tip
         260 loop
         262 non-conductive void
         264 central inductive element
         266 arms
   318 instrument tip
      320 active tip
         368 electrically lengthening sections
         370 capacitive track
         372A,B pair of tracks
         374A,B pair of branches
   418 instrument tip
      420 active tip
         476 deflections
         478 square wave
   518 instrument tip
      520 active tip
         580 elongate track
         582 proximal region
         584 distal region
         586 enlarged proximal end
         588 narrow straight section
         590A,B,C,D deflections
         592 peripheral section
         594 lengthening section
         596 gap
         598 terminal end
   618 instrument tip
      620 active tip
         688 straight track
   718 instrument tip
      720 active tip
         722 planar body
         724 conductive element
         738 central zone
         740 proximal zone
         742 distal zone
         748 conductive plate
         760 conductive loop
         762 non-conductive void

1. An electrosurgical instrument for applying radiofrequency (RF) electromagnetic (EM) energy or microwave frequency EM energy to biological tissue, the instrument comprising:
an instrument tip comprising a planar body separating a first conductive element on a first surface thereof from a second conductive element on a second surface thereof, the second surface facing in an opposite direction to the first surface;
a coaxial feed cable comprising an inner conductor, an outer conductor coaxial with the inner conductor and a dielectric material separating the inner and outer conductors, the coaxial feed cable being for conveying a working signal comprising an RF signal and/or microwave signal;
wherein the inner conductor is electrically connected to the first conductive element and the outer conductor is electrically connected to the second conductive element to enable the instrument tip to receive the working signal,
wherein the first conductive element comprises a pattern configured to set an electrical length of the instrument tip to be greater than an electrical length of a reference instrument tip, and
wherein the reference instrument tip comprises a reference planar body separating a reference first conductive element entirely covering a reference first surface thereof from a reference second conductive element entirely covering a reference second surface thereof, and wherein the reference instrument tip is otherwise equivalent to the electrosurgical instrument tip, with the reference second surface facing in the opposite direction to the reference first surface, and the reference planar body have a same shape and dimensions as the planar body.

2. The electrosurgical instrument according to claim 1, wherein the instrument tip is configured as a half wave resonator, optionally wherein the instrument tip is configured to resonate at one of a following predetermined frequencies: 915 MHz, 2.45 GHz, 5.8 GHz, 14.5 GHz, 24 GHz.

3. The electrosurgical instrument according to claim 1, wherein the instrument tip comprises four quarters along its length, a middle two quarters being combined together to form a central zone, and wherein the pattern forms at least one inductive element in the central zone.

4. The electrosurgical instrument according to claim 3, wherein the inductive element comprises a first conductive region having a width being less than or equal to 90% of a maximum width of a second conductive region of the first conductive element, the second conductive region being located in a distal zone or a proximal zone.

5. The electrosurgical instrument according to claim 1, wherein the pattern includes a lengthening section configured to increase a physical length of the first conductive element relative to a physical length of the planar body.

6. The electrosurgical instrument according to claim 5, wherein the lengthening section has an undulating shape.

7. The electrosurgical instrument according to claim 5, wherein the lengthening section includes one or more branches extending along a respective one or more peripheral edges of the planar body.

8. The electrosurgical instrument according to claim 1, wherein the pattern forms at least one capacitive element in a distal or proximal quarter of the instrument tip.

9. The electrosurgical instrument according to claim 8, wherein each capacitive element comprises a conductive region having an area which fills a majority or an entirety of the distal or proximal quarter in which the capacitive element is formed.

10. The electrosurgical instrument according to claim 1, wherein the pattern comprises a conductive loop encircling a non-conductive region.

11. The electrosurgical instrument according to claim 10, wherein the conductive loop is configured to extend along one or more edges of the planar body at a distal zone of the instrument tip.

12. The electrosurgical instrument according to claim 1, wherein the pattern is symmetrical about a longitudinal axis of the instrument tip.

13. The electrosurgical instrument according to claim 1, wherein the second conductive element covers a majority or an entirety of the second surface of the planar body.

14. The electrosurgical instrument according to claim 1, wherein the instrument tip includes a spacer element proximal to the planar body, wherein the inner conductor is electrically connected to the first conductive element by a proximal transmission line which overlies the spacer element, and wherein the spacer element is chamfered.

15. The electrosurgical instrument according to claim 1, wherein a distal end of the planar body is curved.

16. The electrosurgical instrument according to claim 1, wherein the pattern sets an electrical length of the instrument tip to be greater than or equal to a physical length of the instrument tip.

17. The electrosurgical instrument according to claim 1, wherein the planar body comprises a ferrite material.

18. An electrosurgical instrument for applying radiofrequency (RF) electromagnetic (EM) energy or microwave frequency EM energy to biological tissue, the instrument comprising:
an instrument tip comprising a planar body separating a first conductive element on a first surface thereof from a second conductive element on a second surface thereof, the second surface facing in an opposite direction to the first surface;
a coaxial feed cable comprising an inner conductor, an outer conductor coaxial with the inner conductor and a dielectric material separating the inner and outer conductors, the coaxial feed cable being for conveying a working signal comprising an RF signal and/or microwave signal; and
wherein the inner conductor is electrically connected to the first conductive element and the outer conductor is electrically connected to the second conductive element to enable the instrument tip to receive the working signal,
wherein the first conductive element comprises an elongate track having a peripheral section for conveying the working signal around a periphery of a distal region of the instrument tip for contacting tissue.

19. The electrosurgical instrument of claim 18, wherein the peripheral section has a width of less than 30% of a maximum width of the distal region, optionally less than 20%, optionally less than 10%.

20. The electrosurgical instrument according to claim 18, wherein the peripheral section extends around at least a distal 30% of the planar body, optionally at least a distal 40% of the planar body, optionally at least a distal 50% of the planar body.

21. The electrosurgical instrument of according to claim 18, wherein the elongate track includes a proximal section for connecting the coaxial feed cable to the peripheral section, wherein the proximal section is set back from one or more lateral edges of the planar body in a proximal region of the planar body.

22. The electrosurgical instrument according to claim 18, wherein the elongate track further includes a lengthening section comprising one or more deflections to further increase a physical length of the elongate track relative to a physical length of the planar body.

23. The electrosurgical instrument according to claim 22, wherein the lengthening section is located in the distal region of the instrument tip

24. The electrosurgical instrument according to claim 23, wherein the peripheral section and lengthening section together form a spiral at the distal region of the instrument tip.

25. The electrosurgical instrument of according to claim 18, wherein the first conductive element is configured to match an impedance of the coaxial feed cable.

26. An electrosurgical instrument for applying radiofrequency (RF) electromagnetic (EM) energy or microwave frequency EM energy to biological tissue, the instrument comprising:
an instrument tip comprising a planar body separating a first conductive element on a first surface thereof from a second conductive element on a second surface thereof, the second surface facing in an opposite direction to the first surface;
a coaxial feed cable comprising an inner conductor, an outer conductor coaxial with the inner conductor and a dielectric material separating the inner and outer conductors, the coaxial feed cable being for conveying a working signal comprising an RF signal and/or microwave signal;
wherein the inner conductor is electrically connected to the first conductive element and the outer conductor is electrically connected to the second conductive element to enable the instrument tip to receive the working signal,
wherein the instrument tip further includes a spacer element proximal to the planar body, wherein the spacer element is chamfered.
